(19) United States
(12) Patent Application Publication
(10) Pub. No.: US 2017/0076040 A1
(43) Pub. Date: Mar. 16, 2017

(54) SINGLE-CELL ANALYSIS AS A SENSITIVE AND SPECIFIC METHOD FOR EARLY CANCER DETECTION

(71) Applicants: Tim Hui-Ming Huang, San Antonio, TX (US); Chun-Liang Chen, Helotes,, TX (US); Jeseph Liu, San Antonio, TX (US); Chiou-Miin Wang, Helotes, TX (US); Ian M. Thompson, San Antonio, TX (US); Chun-Lin Lin, San Antonio, TX (US)

(72) Inventors: Tim Hui-Ming Huang, San Antonio, TX (US); Chun-Liang Chen, Helotes, TX (US); Joseph Liu, San Antonio, TX (US); Chiou-Miin Wang, Helotes, TX (US); Ian M. Thompson, San Antonio, TX (US); Chun-Lin Lin, San Antonio, TX (US)

(73) Assignee: THE BOARD OF REGENTS OF THE UNIVERSITY OF TEXAS SYSTEM, Austin, TX (US)

(21) Appl. No.: 15/124,796

(22) PCT Filed: Mar. 14, 2015
(86) PCT No.: PCT/US15/20629

§ 371 Date: Sep. 9, 2016

Related U.S. Application Data
(60) Provisional application No. 61/953,058, filed on Mar. 14, 2014.
(60) Provisional application No. 62/088,573, filed on Dec. 6, 2014.

(51) Int. Cl. G06F 19/26; C12Q 1/68

(52) U.S. Cl. CPC G06F 19/26; C12Q 1/6886; C12Q 2600/158

(57) ABSTRACT

Certain embodiments are directed to methods of measuring single cell levels of biomarkers associated with prostate cancer.

### STATEMENT REGARDING FEDERALLY FUNDED RESEARCH

This invention was made with government support under CA113001 awarded by the National Institutes of Health. The government has certain rights in the invention.

### BACKGROUND

Prostate cancer is the second leading cause of cancer related death for men in USA. Based on rates between 2007 and 2009, 16.2% of men will be diagnosed with prostate cancer during their lifetime. The cost of prostate cancer care was $11.85 billion in 2010. In order to improve the survival rate and alleviate the medical burden, sensitive and specific methods for early detection and effective therapeutics are needed.

The current diagnosis of prostate cancer relies primarily on increased prostate specific antigen (PSA) in the blood and abnormal digital rectal examination (DRE). These two methods have limits on sensitivity and specificity for the detection of prostate cancer. The sensitivity for PSA and DRE as a screening test for prostate cancer was 72% and 53% and the specificity was 93% and 84%, respectively. Positive predictive value was 32% for PSA and 21% for digital rectal examination. Thus, approximately four men with elevated PSA levels undergo prostate biopsies to find one with cancer, and some cancerous men with “normal” PSA levels escape detection using PSA/DRE methods.

Thus, there remains a need for additional methods for detecting prostate cancer with increased sensitivity and specificity as compared to PSA and DRE methods.

### SUMMARY

The studies described herein provide a conceptual advance for deciphering inter-clonal heterogeneity of a tumor. Presently, expression profiles of microdissected tissue are commonly used to stratify cancer subtypes (Tamura et al., Cancer Res 67, 5117-25 (2007)). This kind of analysis is conducted under the assumption that uniform gene expression is present in a cell population. Nevertheless, clonal heterogeneity is increasingly detected in primary tumors (Meacham and Morrison, Nature 501, 328-337 (2013)), and novel approaches are needed to analyze gene expression complexity for risk assessment. The reductionist approach described herein has led to the establishment of a binary code system for single-cell analysis. Interestingly, this binary behavior could not be observed when prostate tumors were analyzed in aggregate in a TCGA cohort. While three of six genes identified using the described methods—TRGBR2, GATA3, and CDKN1C, are known tumor suppressors, their up-regulation has also been reported in advanced cancers (Levy and Hill, Cytokine Growth Factor Rev 17, 41-58 (2006)). Irrespective of their tumorigenic roles, these genes display dichotomous expression patterns that can readily be used for clonal analysis of single cells.

Genes whose complex expression patterns can be reduced to numeric codes for disease diagnosis can be selected from the pool of known biomarkers or potential biomarkers. In certain aspects new biomarkers may also be identified using the methods described herein. While biomarker expression alterations may not directly contribute to a disease process per se, the genes represent a new class of single-cell binary biomarkers. Thus, the “liquid biopsy” or DIGITAL BIOPSY™ described here have broad applications for detecting rare disease cells isolated from bodily fluids, including blood, saliva, breast milk, vaginal secretions, and washes or leftover materials from biopsy needles and surgical blades.

Certain embodiments are directed to methods for assessing and/or detecting a disease or condition by single cell analysis. The single-cell approach described herein reduces the possibility of false positives and false negatives. To that end, the methods would assist in early detection of disease or condition (e.g., prostate cancer), improve human health, and decrease unnecessary medical expenses. The invention utilizes much less invasive methods, for example urine samples can be collected. In certain aspects the urine samples are collected post-DRE. In certain aspects the methods describe herein can be used in combination with known methods of detection or diagnosis, for example in prostate cancer screening methods described herein can be used with other prostate cancer screening methods such as PSA levels in the blood.

The methods described herein are less invasive and use body fluids into which target cells are shed. The target cell can be a diseased or pathogenic cell such as a cancer cell. In certain aspects the body fluid can be blood, cerebrospinal fluid (CSF), saliva, urine, semen, etc. In certain aspects body fluid samples are collected after a procedure that may increase shedding of a target cell into the body fluid. In a further example urine samples can be collected post-DRE.

A sufficient number of prostate cells are found in urine, particularly after DRE, for conducting single cell analysis. In certain embodiments the biological sample need not be a fluid sample, but can be a solid sample that is subsequently dispersed, e.g., a biopsy or fecal sample. In certain aspects the biological sample can be a biopsy or other tissue sample. A tissue sample can be treated with various enzymes that degrade extracellular components and free individual cells from the tissue for analysis.

Single-cell analysis can be used to assess and/or measure biomarkers associated with a disease or pathological condition. In certain aspect cell type specific markers can be used to identify a target cell in a sample. Cell type specific markers are those proteins that are selectively expressed by a tissue or cell type, e.g., prostate cell, colon cell, liver cell, heart cell, lung cell, etc. A number of such markers are known. In a further aspect, disease or pathology related biomarkers can be used to characterize a particular cell. In the example provided here, prostate cells found in a urine sample are analyzed. In certain aspects a urine sample is collected from a patient. In a further aspect the patient had undergone DRE within the last 1, 2, 3, 4, 5, 6, 7, 8, 9, 10, 11, 12, 13, 14, 15, 16, 17, 18, 19, 20, 21, 22, 23, or 24 hours. In other methods a non-urine body fluid is collected or a tissue sample is dispersed for single cell analysis.

Certain embodiments include methods for single cell analysis. Single-cell analysis profiles can provide greater sensitivity and specificity than traditional methods, allowing earlier and more reliable diagnosis of a disease or condition, e.g., prostate cancer. In certain aspects cells are fixed and/or stabilized upon collection and/or isolation. In a further aspect single cells are sorted or selected. Single cells can be sorted manually or by automated sorting or selection. In certain aspects single cells are sorted using a DEPArray or similar technique/instrument. In other aspects single cells are sorted manually by manipulation with a micromanipulator. In further aspects a target cell is identified by cell type specific marker(s). A cell type specific marker can include, but is not limited to, one or more of PSA, PSMA, EpCAM, CK7, or CK8. In certain aspects the cell type specific marker is measured or detected and the level and/or presence/absence of biomarkers is determined.

In certain aspects a cell type can be identified by which proteins it expresses or does not express. For example a particular marker can be expressed for a number of cell types being derived from a common precursor and specific cell types can then be identified using one or more second markers to further classify the general cell type. In certain aspects analysis of urine can be done in conjunction with methods for identifying a particular cell type. Once the particular cell type is identified and isolated other biomarkers can be assessed to characterize each isolated cell. A number of isolated cells are analyzed to obtain a population of characterized cells. In certain aspect the character of the population of characterized cells can be used to determine the diagnosis and/or prognosis of a subject. In a further aspect such a method can be used for assessing cell type character in the blood and detecting and characterizing circulating target cells, such as tumor cells, as a diagnostic or prognostic for cancers or metastatic cancers.

Certain embodiments are directed to monitoring a subject over time. Biological samples can be obtain 1, 2, 3, 4, 5, 6, 7, 8, 9, 10, or more times over 1, 2, 3, 4, 5, 6, 7, 8, 9, 10 or more days, weeks, months, or years. For example urine, blood, or other body fluids as well as tissue samples can be obtained over time. The method can be used to monitor patients that are at risk for disease development, such as prostate cancer development or progression. Patients can include patients undergoing therapy/surgery and post-therapy/surgery. A subject can be at risk for disease development based on family history, genomic marker of predisposition, and/or physiologic symptoms that indicate a risk for disease development.

Certain embodiments are directed to methods of detecting prostate cancer cells comprising (a) measuring levels of a biomarker in a single prostate cell isolated from post-digital rectal examination (DRE) urine of subjects; and (b) comparing the single cell levels of the biomarker to a reference to classify the prostate cell as cancerous or non-cancerous. In certain aspects a prostate cell is selected by using a tissue specific marker. A prostate specific marker can be prostate specific antigen (PSA) and/or prostate specific membrane antigen (PSMA). In a further aspect a prostate specific marker is EpCAM and/or CK7/8. In still other aspects the prostate specific marker is PSA, EpCAM, and CK7/8. In certain aspects a biomarker is CXCL6, TGFBR2, GSK3B, CDKN1C, GATA3 and EIF4EBP1. In further aspects a single prostate cancer cell is isolated using a dielectrophoresis cage array, a microfluidic device, or micromanipulation.

Certain embodiments are directed to methods for detecting prostate cancer cells in a urine sample comprising: (a) concentrating cells in a urine sample; (b) contacting the concentrated cells with a detectable antibody that binds a prostate cell specific marker; and (c) conducting biomarker profiling on the prostate cell. In certain aspects the cell specific marker is prostate specific antigen (PSA) or prostate specific membrane antigen (PSMA). In further aspects the prostate specific marker is EpCAM and/or CK7/8. In still further aspects the cell specific marker is PSA, EpCAM, and CK7/8.

Other embodiments are directed to methods for expressing complex gene expression patterns as binary code strings comprising: identifying and ordering a plurality biomarkers that individually or in combination correlate with a pathological state into a binary code string that is correlated with a diagnosis or prognosis, wherein the biomarkers are genes that exhibit bimodal expression. In certain aspects the biomarkers comprise the genes CXCL6, TGFBR2, GSK3B, CDKN1C, GATA3 and EIF4EBP1. In certain aspects the binary code strings are composed of a 0 representing low expression or 1 representing high expression for each gene.

Certain embodiments are directed to a computer implemented method comprising the steps of (a) obtaining single cell protein level measurements of one or more biomarker, (b) transforming the obtained measurements to a score or ratio, and (c) determining if the measurements indicate the presence of prostate cancer.

Other embodiments include methods of treating a patient having prostate cancer comprising: administering a treatment for prostate cancer to a patient having elevated single cell levels of one or more biomarker.

Certain embodiments are directed to methods of monitoring a subject comprising: (a) measuring levels of a biomarker in a single prostate cell isolated from post-digital rectal examination (DRE) urine of subjects periodically; and (b) comparing the single cell levels of the biomarker to a reference to classify the prostate cell as cancerous or non-cancerous over time. In certain aspects the subject has prostate cancer, is at risk of developing prostate cancer, or is undergoing prostate cancer treatment.

Further embodiments are directed to methods for determining a biomarker profile of a population of representative cells isolated from urine comprising: (a) contacting cells isolated from urine with a detection agent that identifies a population of representative cells in the sample; (b) isolating the identified cells as single cell isolates; (c) conducting biomarker analysis on the each of the isolated single cells to determine a biomarker profile.

Embodiments include methods for determining a biomarker expression profile for detecting and evaluating prostate cancer in a patient comprising: (a) contacting cells isolated from urine obtained from a patient suspected of having prostate cancer with a detection agent that identifies a population of prostate cells in the sample; (b) isolating the identified prostate cells as single cell isolates; (c) conducting prostate cancer biomarker analysis on the each of the isolated single cells to determine a biomarker profile; (d) assessing the biomarker profiles of a plurality of prostate cells and providing an assessment of the patient relating to a diagnosis of prostate cancer or a prognosis for prostate cancer.

Further embodiments include methods for display of a biomarker expression profile comprising: (a) obtaining single cell biomarker profiles for a plurality of target cells isolated from a sample; (b) grouping the single cell biomarker profiles into two or more pathological stages or states based on correlation of the single cell biomarker profile with a normal, benign, or pathological condition; and (c) displaying geometric shapes representing various biomarker profiles, wherein the geometric shape has a size that is proportional to number of cells having a particular profile and an indicator of which state the single cell biomarker profile correlates. In certain aspects the geometric shape is a circle with the radius or diameter of the circle being proportional to the number of binary clones or codes identified in a cell population. In certain aspects the cell with the most developed pathological character can be represented by a red color with a normal cell type being represented by a more subdued color such as green or a pale shade of blue, etc.

The term “isolated” can refer to a cell, nucleic acid, or polypeptide that has had some or substantially all of the non-cellular material (e.g., other components of a biological fluid, extracellular matrix, tissue scaffold, etc.), cellular material, bacterial material, viral material, or culture medium (when produced by recombinant DNA techniques) of their source of origin.

Moieties of the invention, such as oligonucleotides, polypeptides, peptides, antigens, or immunogens, may be conjugated or linked covalently or noncovalently to other moieties such as adjuvants, proteins, peptides, supports, fluorescence moieties, or labels. The term “conjugate” or “immunoconjugate” is broadly used to define the operative association of one moiety with another agent and is not intended to refer solely to any type of operative association, and is particularly not limited to chemical “conjugation.”

The phrase “specifically binds” or “specifically immunoreactive” to a target refers to a binding reaction that is determinative of the presence of the molecule in the presence of a heterogeneous population of other biologics. Thus, under designated immunoassay conditions, a specified molecule binds preferentially to a particular target and does not bind in a significant amount to other biologics present in the sample. Specific binding of an antibody to a target under such conditions requires the antibody be selected for its specificity to the target. A variety of immunoassay formats may be used to select antibodies specifically immunoreactive with a particular protein. For example, solid-phase ELISA immunoassays are routinely used to select monoclonal antibodies specifically immunoreactive with a protein. See, e.g., Harlow and Lane, Antibodies: A Laboratory Manual, Cold Spring Harbor Press, 1988, for a description of immunoassay formats and conditions that can be used to determine specific immunoreactivity.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIGS. 1A-1C. Urine sample analysis using DEPArray after immunostaining shows heterogeneous PSA expression profiles of cells from urine samples of prostate cancer patients. (A) A flow chart illustration shows the urine sample analysis protocol. (B) Immunostaining of EpCAM, CK7/8, and PSA on representative PSAlow and PSAhigh cells from a urine sample. (C) Bar graphs display PSA levels of triple-positive (EpCAM-CK7/8-PSA) cells from urine samples of two prostate cancer patients and one normal individual.

FIG. 2. Scatter plots show expression profiles of PSA and EpCAM on cells from urine samples of one prostate cancer patient and one BPH patient using DEPArray.

FIG. 3. PSA/PSMA expression patterns in urine samples.

FIG. 4. Diagram of computer implemented aspects of the invention.

FIGS. 5A-5D. Gene expression analysis of urinary prostate cells. (a), Exfoliated prostate cells isolated from urine sediment were positively identified by fluorescent markers PSA (red dye) and PSMA (green dye) for single-cell isolation. (b), Representative examples (#25 and N02) of microfluidic PCR analysis of KLK3 and UBB genes. Ct (threshold cycle) value was the outcome of RT-PCR analysis for fold changes of gene expression. ΔRN: Normalized Reporter=fluorescence intensity of reporter dye divided by that of reference dye. (c), Expression profiles of PPAP2A in 1220 single cells (red hair lines) isolated from normal controls (Ctrl) and patients with benign prostate hyperplasia (BPH), high-grade prostatic intraepithelial neoplasia (HGPIN), and prostate cancer (PCa). Additional single-cell expression profiles are presented in (d), Dichotomous single-cell expression profiles of six genes. Lower panel: A violin graph combining a box plot with a kernel density plot displays a bimodal expression pattern, 0 for low and 1 for high expression, for a given gene in a total of 1220 cells analyzed. Normalized expression values range from 0 to 35.

FIGS. 6A-6B. Stepwise Conversion of Bimodal Expression Data to Binary Code Format for Clonal Analysis (A) Step 1: Kernel density plots of gene expression (e.g., CXCL6 and TGFBR2) in 1220 exfoliated prostate cells. Step 2: In parallel coordinate plots 1220 cells were visualized as dots and aligned horizontally according to expression of CXCL6 and TGFBR2. Concordant lines (brown) connected the expression dots of two genes for all the cells. Step 3: The expression levels (−ΔΔCt) of the genes higher than the cutting points (black dash line) derived from Kernel density estimates were coded into 1, whereas lower than the cutting points, were decoded into 0. Step 4: The path of four cells (patina lines in the plots) was highlighted to illustrate four possible combinatory codes: 00, 01, 10, and 11. Step 5: The 6-gene binary codes were constructed for hypothetical cell X (000100) and cell Y (111011) as examples. Step 6: Parallel coordinate plots for cell X and cell Y. (B) All 6-gene binary codes (in the order of CXCL6, TGFBR2, GSK3B, CDKN1C, GATA3, and EIF4EBP1) for the prostate cells from N #01 and Pt #40.

FIG. 7. Polar Plot Analysis of 64 Combinatorial Codes in Normal Controls and Patient Groups Polar plots display simulated and existing proportions of each of 64 CANPs in exfoliated prostate cells derived from normal controls (Normal), benign prostate hyperplasia (BPH), Lowrisk group prostate cancer (including high-grade prostatic intraepithelial neoplasia), and High risk group prostate cancer patients. The value of proportions increases as the distance away from the center increases in the plots. From 2000 simulations the average proportions of 64 CANPs are connected as solid lines with 1.7 times standard deviations shown in colorful shades (green in Normal, blue-violet in BPH, purple in Low-risk group, and red in High-risk group). The asterisks are the corresponding existing proportions.

FIG. 8. Gene expression analysis of urinary prostate cells in patient subgroups. Microfluidic PCR analysis was conducted in 1220 cells from normal controls, patients with benign prostate hyperplasia (BPH), high-grade prostatic intraepithelial neoplasia (HGPIN), and prostate cancer subgroups (PCa-I, -II, and -III). Normalized expression values range from 0 to 35. Representative examples of gene expression are shown here. Violin plots (bottom) display expression distribution patterns and median values of cells in control and patient subgroups. *P<0.05, **P<0.01, ***P<0.001.

FIG. 9. Illustration of methods for establishing biomarkers for use in single cell biomarker profiling.

FIG. 10. Prostate-specific Single Cells Isolated from Urine Samples. (A) Illustration of urine collection and representative prostate-specific antigen (PSA)/prostatespecific membrane antigen (PSMA)-positive and -negative cells exfoliated in urine sediments using the DEPArray System™. (B) Expression levels of PSA and PSMA in tissues from prostate and other organs. PSA (KLK3) data were taken from Normal Tissue Database in Oncogenomics (available on the internet at home.ccr.cancer.gov/oncology/oncogenomics/). PSMA (FOLH1) data were derived from online data base in GeneCards (available on the worldwideweb at genecards.org/) and BioGPS (available on the internet at biogps.org/#goto=welcome). (C) PSA expression shown in fluorescent intensity among urinary exfoliated prostate-specific cells isolated from four patients using DEPArray. (D) A schematic of single-cell collection using a micromanipulator and micropipette and microfluidic RT-qPCR. Exfoliated prostate cells were positively identified by fluorescent antibodies against PSA (Cy3, red) and PSMA (APC, cyan). Representative expressions of KLK3 and UBB genes of prostate cells from two urine samples (#25 and N02) were presented. ΔRN: relative gene expression.

FIG. 11. Gene Expression Patterns of Single Exfoliated Prostate Cells. (A) Single-cell expression profiles of UBB in a total of 1220 single cells (represented by vertical lines) isolated from normal controls (Ctrl), patients with benign prostate hyperplasia (BPH), high-grade prostatic intraepithelial neoplasia (HGPIN), and prostate cancer (PCa). UBB expression of cells from the same patient was arranged in a gray horizontal frame, from the lowest level (left) to the highest (right). The expression level in UBB was displayed in Ct values. (B) and (C) are same as in (A), but the expression values range from 0 to 35 (−ΔΔCt). (D) Single-cell expression profiles (represented by red vertical lines) for selected genes in 1220 exfoliated prostate cells analyzed using Kernel density estimation (black curves). Genes with bimodal expression patterns are shown in the left panel compared to the other expression patterns on the right panel.

FIG. 12. Stepwise Conversion of Bimodal Expression Data to Binary Code Format for Clonal Analysis. (A) Step 1: Kernel density plots of gene expression (e.g., CXCL6 and TGFBR2) in 1220 exfoliated prostate cells. Step 2: In parallel coordinate plots 1220 cells were visualized as dots and aligned horizontally according to expression of CXCL6 and TGFBR2. Concordant lines connected the expression dots of two genes for all the cells. Step 3: The expression levels (−ΔΔCt) of the genes higher than the cutting points (black dash line) derived from Kernel density estimates were coded into 1, whereas lower than the cutting points, were decoded into 0. Step 4: The path of four cells (patina lines in the plots) was highlighted to illustrate four possible combinatory codes: 00, 01, 10, and 11. Step 5: The 6-gene binary codes were constructed for hypothetical cell X (000100) and cell Y (111011) as examples. Step 6: Parallel coordinate plots for cell X and cell Y. (B) All 6-gene binary codes (in the order of CXCL6, TGFBR2, GSK3B, CDKN1C, GATA3, and EIF4EBP1) for the prostate cells from N #01 and Pt #40. Any binary codes shared two cells or more were highlighted.

FIG. 13. Polar Plot Analysis of 64 Combinatorial Codes in Normal Controls and Patient Groups. Polar plots display simulated and existing proportions of each of 64 CANPs in exfoliated prostate cells derived from normal controls (Normal), benign prostate hyperplasia (BPH), Lowrisk group prostate cancer (including high-grade prostatic intraepithelial neoplasia), and High risk group prostate cancer patients. The value of proportions increases as the distance away from the center increases in the plots. From 2000 simulations the average proportions of 64 CANPs are connected as solid lines with 1.7 times standard deviations shown in colorful shades (green in Normal, blue-violet in BPH, purple in Low-risk group, and red in High-risk group). The asterisks are the corresponding existing proportions.

FIG. 14. Clonally-Associated Numerical Patterns (CANPs) in Normal Controls and Different Patient Groups (A) Clonal profiles of normal controls and patients portrayed using CANPs. Class A, B, C, and D contained unique CANPs in normal controls (Normal) and benign prostate hyperplasia (BPH), low-risk group prostate cancer (including high-grade prostatic intraepithelial neoplasia), and high-risk group prostate cancer groups, respectively. Class E: CANPs that cannot differentiate exfoliated cell clones between these groups. Other combinatorial codes observed only in single cells in each patient were classified in Class F. Clonal profile of each patient was presented in different colors of the classes (green for Class A, light blue for Class B, pink for Class C, red for Class D, orange for Class E, and light green for Class F). The size of the dots represents the size of clonal cells. (B) Composition of the classes in low-risk group (n=335) and high-risk group (n=362). The classes shared the same CANPs as those in (A).

FIG. 15. Phenotypic Analysis of Two Prostate Cancer Cell Lines Carrying Respective CANPs 000000 and 111011. (A) Expression profiles of six bimodal genes in LNCaP and PC3 cells treated with and without R1881 (1 nM). Bars show the mean±S.D. of the expression levels (fold changes). P values were obtained using two-tailed Student's t-test for the comparison between LNCaP and PC3 cells in all time points. (B) Growth curves in proliferation, migration, and invasion assays measured by IncuCyte for LNCaP and PC3 cells in the presence and absence of R1881. The confluence values in the proliferation assay were normalized to that of a specific cell-type by the average percent confluence at time=0. Each line represents the average growth from nine technique repeats (mean±S.D.). The result was independently validated in other sample. Migration and invasion data were derived from independent experiments of three samples for each cell line (mean±S.D.). P values were obtained using two-way ANOVA for the comparison among values of four treated groups. (C) RT-qPCR of EMT gene panel in LNCaP and PC3 cells treated with and without R1881. Bars show the mean±S.D. of the expression levels (fold changes). P values were obtained using two-tailed Student's t-test for the comparison between LNCaP and PC3 cells in all time points. (D) RT-qPCR of TGFBR2 and GSK3B genes in PC3 knockdown cells. P values were obtained using two-tailed Student's t-test. (E) Growth curves in proliferation assay of PC3 cells with TGFBR2 and GSK3B knockdown. The confluence values in the proliferation assay were normalized to that of a specific cell-type by the average percent confluence at time=0. Each line represents the average growth from twelve technique repeats (mean±S.D.). The result was independently validated in other sample. p values were obtained using two-way ANOVA for the comparison among values of the control and two knockdown groups. Migration and invasion assays of PC3 with TGFBR2 and GSK3B knockdown. Bars show the mean±S.D. from three independently biological repeats. *p<0.05, **p<0.01, ***p<0.001.

FIG. 16. Genes with Different Expression Distribution Shown in Single-Cell Profiles. Microfluidic PCR analysis conducted in a total of 1220 cells (represented by red vertical lines) from normal controls (Ctrl), patients with benign prostate hyperplasia (BPH), highgrade prostatic intraepithelial neoplasia (HGPIN), and prostate cancer (PCa) patients. Cells from the same patient were displayed within the same horizontal frame, with lowest expression level on the left to the highest on the right. Kernel density estimation (bottom) displays smooth expression distribution for each gene.

FIG. 17. Parallel Coordinates Plots and Corresponding Combinatorial Codes of 33 Patients and 5 Normal Controls. Single-cell expression patterns of genes are connected in a string (brown) for a given cell. A total of 1220 connected lines are shown here. A patina line traces an expression path of a cell across six genes. Connectivity paths are converted to binary code-strings with 0 for low and 1 for high expression, respectively. Clonally-associated numerical patterns (CANPs) are highlighted in different colors corresponding to classes in FIG. 5 (A): green for Class A (n=7), light blue for Class B (n=7), pink for Class C (n=16), red for Class D (n=8), and orange for Class E (n=12).

FIG. 18. A Schematic of Resampling Process and Simulation Analysis in 832 Clonal Cells for Identification of Clonally-Associated Numerical Patterns (Using the Normal Control Group as an Example). (A) Two-layer resampling method applied for generating simulation groups. The 1st layer was to decide the source of cells. Randomly choose a group X from four existing groups: normal control (NC), benign prostate hyperplasia (BPH), low-risk prostate cancer, and highrisk prostate cancer. The 2nd layer was to pick up cells. Randomly choose a cell from the selected group in the previous layer. The number of cells in the pool differed by groups (67 in NC, 68 in BPH, 335 in low-risk, and 362 in high-risk). The same process was repeated until 67 cells were sampled for a simulation group. (B) The same resampling process in (A) was repeated to generate 2000 simulation groups. (C) Use the means and standard deviations (S.D.) to construct polar plots. Means are displayed as the solid line and 1.7×S.D. as the shadow span. The asterisk symbols denote the proportion in the cells from the existing normal control group.

FIG. 19. A Schematic Resampling Process and Euclidean Distance Analysis in 832 Clonal Cells in two Groups (Using Normal Control (NC) and Benign Prostate Hyperplasia (BPH) Groups as Examples). (A) Two-layer resampling method applied for generating simulation groups. Same as in FIG. 18A, but the same process was repeated until 67 cells in the simulation group of normal control (SG.NC) and 68 cells in the simulation group of BPH (SG.BPH) were sampled. (B) The same resampling process in (A) was repeated to generate 5000 pairs of simulation groups. (C) Use the values of Euclidean distance from 5000 simulations to construct an experimental distribution. The Euclidean distance of the cells in the existing normal control and BPH groups is pointed by an arrow.

FIG. 20. Euclidean Distance Analysis of 38 Clonally-Associated Numerical Patterns Normal Control (Normal), Benign Prostate Hyperplasia (BPH), Low-Risk Prostate Cancer (Including High-grade Prostatic Intraepithelial Neoplasia), and High-Risk Prostate Cancer Groups. Experimental distribution of Euclidean distance was constructed based on 5000 simulations in any two groups. Red arrows indicated the observed Euclidean distance calculated from the existing groups. The p values of the observed Euclidean distance in six pairs of comparison are all less than 0.0001 in one-tailed probability test.

FIG. 21. Androgen Inhibited the Expression of CXCL6 and CDKN1C in LNCaP. Expression profiles of CXCL6 and CDKN1C in LNCaP cells treated with and without R1881 (1 nM). Bars show the mean±S.D. of the expression levels (fold changes). P values were obtained using two-tailed Student's t-test for the comparison in two time points. ***p<0.001.

### DESCRIPTION

The use of single cell analysis of prostate cancer patient urine samples improves the sensitivity and specificity for prostate specific antigen (PSA) and DRE screening for early prostate cancer diagnosis. The current diagnosis of prostate cancer relies primarily on increased blood prostate specific antigen (PSA) and abnormal digital rectal examination (DRE). These two methods have limits on sensitivity and specificity for the detection of prostate cancer. Evaluation of PSA level in the serum is an indirect and secondary measurement of elevated PSA in the prostate cancer. An inherent limitation of DRE is that only 85 percent of cancers arise peripherally where they can be detected with a finger examination. Within a threshold value of 4 ng/ml, around 15% of men will have prostate cancer that goes undetected, most of whom will have potentially curable disease. The false positives and negatives create unnecessary personal anxiety, increase medical expense, and leave cancerous patients untreated.

Certain aspects include one or more steps selected from (a) fixing of urine samples upon their collection, and (b) single-cell analysis of PSA and PSMA expressions on cells in urine using single cell isolation techniques, such as a DEPArray™ (Silicon Biosystems) as a screen tool for detection of prostate cancer. DEPArray™ technology is based on moving dielectrophoresis cages, to individually sort cells out of a suspension of a relatively small number of cells. The system's core is a chip where an array of individually controllable cages of A/C electrical field is formed. Each cell in suspension is trapped into a cage and numbered. Selected cells then can be individually moved and collected through a software calculated pathway.

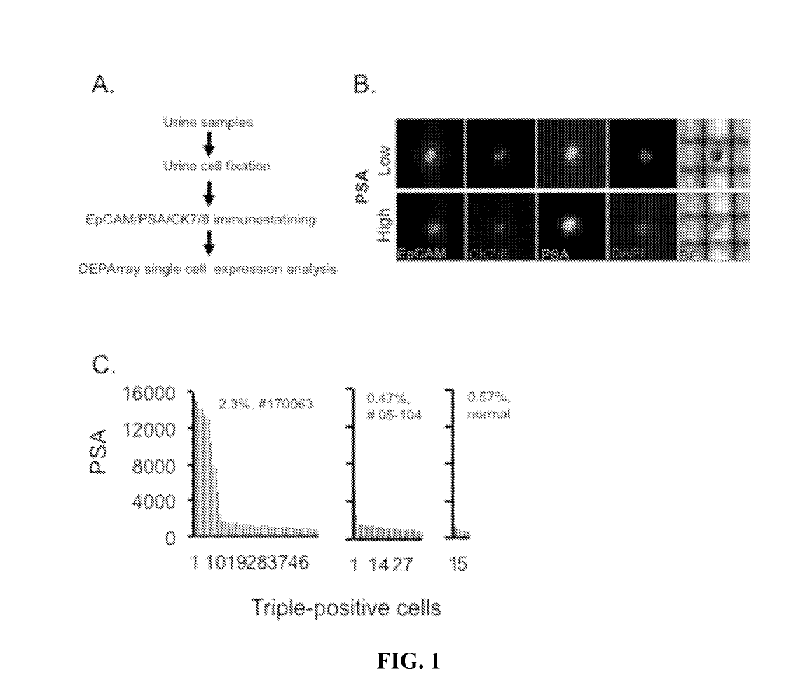

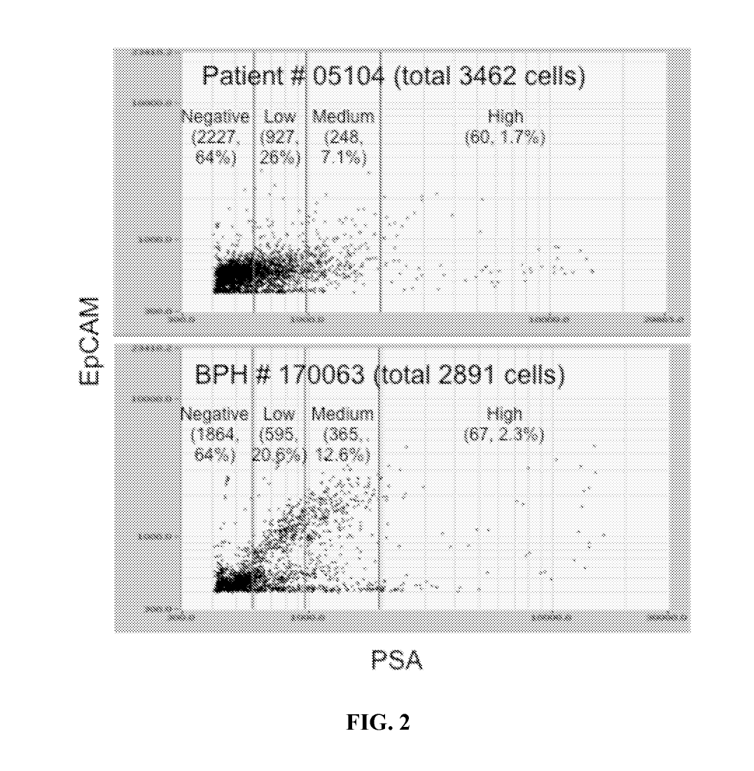

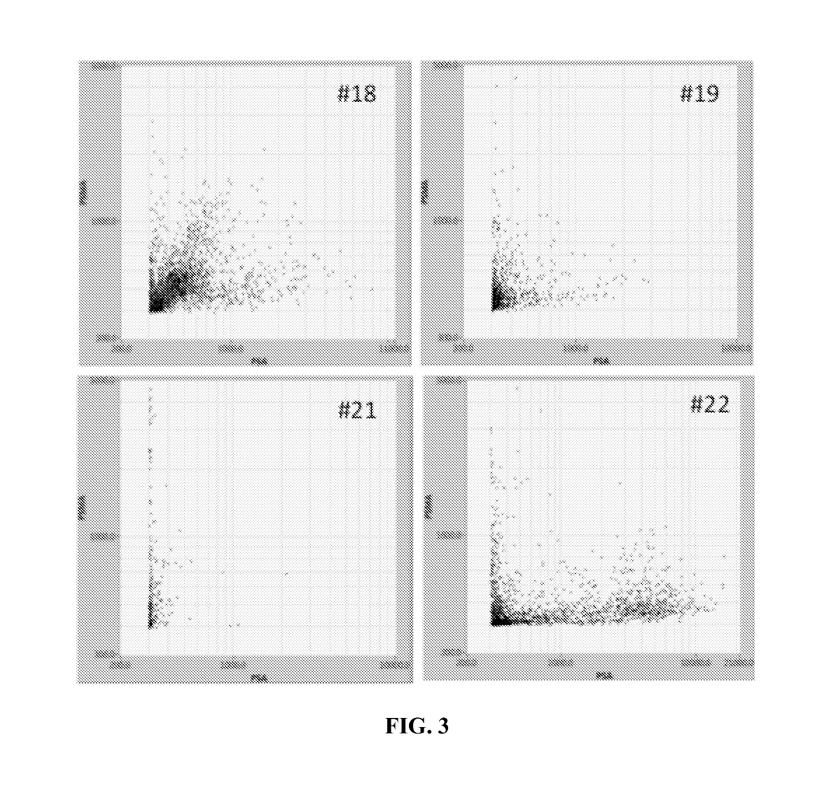

Studies demonstrate that single cells from urine samples with heterogeneous PSA expressions can serve as biomarkers for diagnosis of prostate cancer (FIG. 1 and FIG. 2).

### I. SINGLE CELL ANALYSIS AND DIGITAL BIOPSY™

Isolation of Single Cells.

Biological samples can be collected in a needle, container, syringe, cup, bag, or other suitable collection device. In certain aspects the biological sample is contacted with a preservative. Typically biological samples are cooled (kept on ice or refrigerated) and/or processed immediately. In certain aspects cellular components are precipitated by centrifugation. In certain aspects a tissue sample is dispersed and optionally clarified or filtered prior to centrifugation. After centrifugation the supernatant can be removed leaving a cell pellet in the container. Cell pellets are suspended in a buffer solution and transferred to a second centrifuge tube (e.g., a low-retention centrifuge tube) and spun again to pellet the cells. The wash and centrifugation steps can be repeated multiple times. Cell pellets are suspended in a trypsin containing buffer to dissociate cell aggregates followed by neutralization with an appropriate solution. The neutralized solution is then centrifuged. After the supernatant is removed, cell pellets are suspended in labeling buffer and labeled with one or more primary antibodies that specifically bind to a target cell. The labeled cells are collected and washed to remove unbound primary antibodies. In certain aspects a secondary antibody is provided in an appropriate solution at an appropriate dilution. The cells are collected, e.g., centrifuged and washed with an appropriate buffer to remove the secondary antibody. The cells are suspended in a buffer compatible with immunostaining and examined for immunostaining Single cells identified by a particular antibody binding profile are isolated. In certain aspects the cells can be isolated using a combined micromanipulator-microinjector system (CM2S) (Chen et al. Prostate 73, 813-826 (2013)). The isolated cell is lysed in reaction buffer and either analyzed or stored for later analysis, e.g., frozen.

Single-Cell Microfluidic PCR.

In certain aspects microfluidics based RT-PCR can be used to amplify target nucleic acids. Single-cell microfluidics-based RT-PCR analysis is carried out using appropriate components. A portion a single cell lysate is subjected to PCR amplification using appropriate primers for one or more genes and a control gene. In certain aspects genomic contamination is reduced by incubation of the lysate with DNase I solution. PCR primers of selected genes for expression profiling can be selected from known primer sequences or designed using available computer software. A primer mixture for each panel is prepared in buffer by pooling all the primers of each panel.

Reverse transcription (RT) and pre-amplification are performed on a single-cell total RNA reaction mix comprising a reverse transcriptase and thermocycle DNA polymerase and a primer mix. RT is performed for a selected time period and then inactivated. Pre-amplification follows the RT reaction. Excessive primers in pre-amplification are removed by digestion with an exonuclease. Pre-amplified products can be diluted prior to PCR. The pre-amplified products are subjected to PCR.

A. Single-cell expression data analysis.

Data Normalization.

Expression levels of 35 genes, obtained as threshold cycle (CO values, were normalized to that of the control reference gene UBB and displayed as −ΔΔCₜ values²⁵. The UBB gene was used as a control because its mRNA was found to be highly stable in single prostate cells in our previous microfluidics-based PCR assays¹⁶. We only selected cells that expressed UBB at a threshold of Cₜ≦30 after pre-amplification, assuming that these cells expressing robust expression of UBB are less likely to contain degraded RNA. The −ΔΔCₜ values ranged from the lowest expression level of 0 to the highest expression level of 35, which were used to construct expression heatmaps (see FIGS. 1 and 2 and Supplementary FIG. 2).

Violin Plot Analysis.

A violin expression plot, which combines a box plot and a rotated kernel density plot¹⁷, were constructed for each gene to determine clonal distributions of gene expression in a given population of prostate cells. The density trace is plotted symmetrically to the left and the right the vertical box plot, and there is no difference in these density traces other than the direction in which they extend. Median expression levels of these genes from urinary single cells isolated in 1) normal controls and patients diagnosed with 2) benign prostate hyperplasia (BPH), 3) prostatic intraepithelial neoplasia (PIN) and 4) prostate cancer were analyzed using one-way ANOVA and unpaired Student's t test using R. A P value of <0.05 is considered as statistically significant.

Parallel Coordinate Plot Analysis.

Expression patterns of 6 genes in urinary single cells were visualized in parallel coordinate plots using the software of GGobi data visualization system²⁶. Each parallel coordinate plot was composed of points and lines. The points, referring to cells (total 1,220 cells), were arranged from the left to the right for each gene according to its gene expression values from the least to the highest. The lines linked to these points displayed expression connectivity among these 6 genes. Expression connectivities of selected cells for each patient were highlighted in patina color, and all the rest were in brown color (see explanations in the main text).

In Silico Analysis of Gene Expression.

Gene expression (RNA-seq) data of adjacent normal (n=37) and primary PCa (n=140) used for this study were obtained from The Cancer Genome Atlas (TCGA). In order to display the expression level of selected genes in the same heat map, TCGA data were adjusted using Normalize Genes/Rows function in the software of MultipleExperiment Viewer 4.8. This process standardized gene expression values using the mean and the standard deviation of the row of the matrix to which the gene belongs. The difference between Prostate samples and Normal samples was further compared by Student's t-test using Prism 6 (GraphPad Software, La Jolla, Calif.). A P value of <0.05 is considered as statistically significant.

B. Biopsy Graphical Display

Certain embodiments include the graphical display of analysis of a population of single cells. In certain aspect this graphical display is called DIGITAL BIOPSY™. The graphical methods are used to convey the results of the analysis in a simple easy to read format that has the general appearance of histology section. Steps for preparing such a graphical display include one or more of: (a) Analyzing a population of single cells to select target cells for biomarker profiling. (b) analyzing the selected cells to determine the expression level of components of a biomarker panel (e.g., protein or nucleic acid biomarkers). (c) Quantization of biomarker data to a binary code where “0” or “1” represents gene underexpression or overexpression, respectively. A violin plot can be used to identify appropriate cutoff point for assignment of binary value. (d) Using a parallel coordinate plot (PCP) for visualizing the range of results from a batch of clinical specimens. A particular order of biomarkers are used to represent the binary results for a biomarker panel, which are displayed as a binary clone for a particular cell with a particular binary code, e.g., with a six marker panel a six number binary code is established (e.g., all cells having a binary code 001100 are designated as the same binary clone). For a six marker biomarker panel there are 2⁶=64 potential binary codes/clones. (e) Each unique binary code/clone is quantified and the frequency of detection of each binary code/clone is represented by a colored circle positioned within a boundary. The circle size is proportional to the number of cells detected for a particular binary code/clone. (f) The analysis and graphical depiction of the results correlates to the clinical diagnosis of each individual tested and results in a powerful and easy to interpret display of pathologic significance. The analysis and/or graphical method can be used to test a patient's disease status as well as to monitor a patient over time, as the disease may progress. As an example of the success of the described method, the inventors have identified the specific binary gene expression clones that correlate with more advanced (Stage II and Stage III) prostate cancer vs normal controls, BPH and Stage I.

The graphical display of biomarker panel results can be used for analysis and display of various biomarker panels for diseases including cancer. In certain aspects the method can be used on various clinical specimens such as tissue, blood, urine, serum, saliva, and sweat samples. The only requirement of the sample is that it contains target cells and can be dispersed to include a population of single cell targets.

### II. ANALYSIS AND GRAPHICAL DISPLAY IN PROSTATE CANCER

Prostate cancer is a form of cancer that develops in the prostate, a gland in the male reproductive system. The cancer cells may metastasize (spread) from the prostate to other parts of the body, particularly the bones and lymph nodes. Prostate cancer can cause pain, difficulty in urinating, problems during sexual intercourse, or erectile dysfunction. Other symptoms can potentially develop during later stages of the disease.

Rates of detection of prostate cancers vary widely across the world, with South and East Asia detecting less frequently than in Europe, and especially the United States. Prostate cancer tends to develop in men over the age of fifty. Many factors, including genetics and diet, have been implicated in the development of prostate cancer. The presence of prostate cancer may be indicated by symptoms, physical examination, prostate specific antigen (PSA), or biopsy. There is controversy about the accuracy of the PSA test and the value of screening. Suspected prostate cancer is typically confirmed by taking a biopsy of the prostate and examining it under a microscope. Further tests, such as CT scans and bone scans, may be performed to determine whether prostate cancer has spread.

Treatment options for prostate cancer with intent to cure are primarily surgery, radiation therapy, and proton therapy. Other treatments, such as hormonal therapy, chemotherapy, cryosurgery, and high intensity focused ultrasound (HIFU) also exist, depending on the clinical scenario and desired outcome.

The age and underlying health of the man, the extent of metastasis, appearance under the microscope, and response of the cancer to initial treatment are important in determining the outcome of the disease. The decision whether or not to treat localized prostate cancer (a tumor that is contained within the prostate) with curative intent is a patient trade-off between the expected beneficial and harmful effects in terms of patient survival and quality of life.

### III. METHODS OF DETECTING PROSTATE CANCER

The single-cell approach described herein reduces the possibility of false positives and false negatives. To that end, the methods would assist in early detection of prostate cancer, improve human health, and decrease unnecessary medical expenses. The invention utilizes much less invasive method with the urine samples that are usually collected post-DRE. The methods can be used in combination with other prostate cancer screening methods such as PSA levels in the blood.

The methods described herein are less invasive, e.g., urine samples are collected post-DRE. In combination with PSA in the blood, single cell analysis using post-DRE urine samples can be used for detecting prostate cancer. A sufficient number of prostate cells are found in urine after DRE for conducting single cell analysis.

The method includes one or more of the following steps. Urine samples and/or other biological samples are collected from a subject. In certain aspects the urine sample is collected after DRE. In certain aspects the urine samples are contacted with a preservative. Cells present in the sample are separated from biological fluids. For example, the cells in the sample are pelleted by centrifugation.

The isolated cells are processed. In certain aspects the cells are contacted with a detectable antibody. The antibody or antibodies include antibodies that bind proteins that are used as a control, a reference, or a biomarker. In certain aspects the antibody is detectably labeled. Detectable labeled refers to the attachment of a moiety to the antibody that can be directly or indirectly detected and/or measured.

The labeled cells can then be isolated and/or sorted. In certain aspects the cells are loaded onto a DEPArray™ for single cell isolation and then BioMark™ molecular profiling device using TBIIR and miRNA gene primer panel.

In certain aspects all or a portion of the cells collected from the sample are fixed. For fixed cells, pellets are washed, fixed, and antibody labeled. Cells are fixed using formaldehyde. The fixed cells are labeled with a detectable antibody. The labeled cells are then sorted and/or isolated and analyzed at the single cell level.

In certain aspects the labeled cells are analyzed using a DEPArray™ in conjunction with DEPArray™ data analysis. Several dozens to thousands of cells isolated from urine are loaded unto DEPArray chips (cat# Silicon Biosystems, Inc) according to manufacturer's protocol. For live cells, the cells were suspended in DMEM+5% FBS+P/S (1×) and in SB115 buffer.

### IV. BIOMARKERS

A biomarker is a biomolecule that is differentially present in a sample taken from a subject of one phenotypic status (e.g., having a disease) as compared with another phenotypic status (e.g., not having the disease). A biomarker is differentially present between different phenotypic statuses if the mean or median expression level of the biomarker in the different groups is calculated to be statistically significant. Common tests for statistical significance include, among others, t-test, ANOVA, Kruskal-Wallis, Wilcoxon, Mann-Whitney and odds ratio. Biomarkers, alone or in combination, provide measures of relative risk that a subject belongs to one phenotypic status or another. As such, they are useful as markers for disease (diagnostics), therapeutic effectiveness of a drug (theranostics) and of drug toxicity.

A biomarker panel can include 2, 3, 4, 5, 6, 7, 8, 9, 10, or more biomarkers. In certain aspects the biomarkers are correlated to particular state, such as normal, benign, or varying degrees of a pathological state.

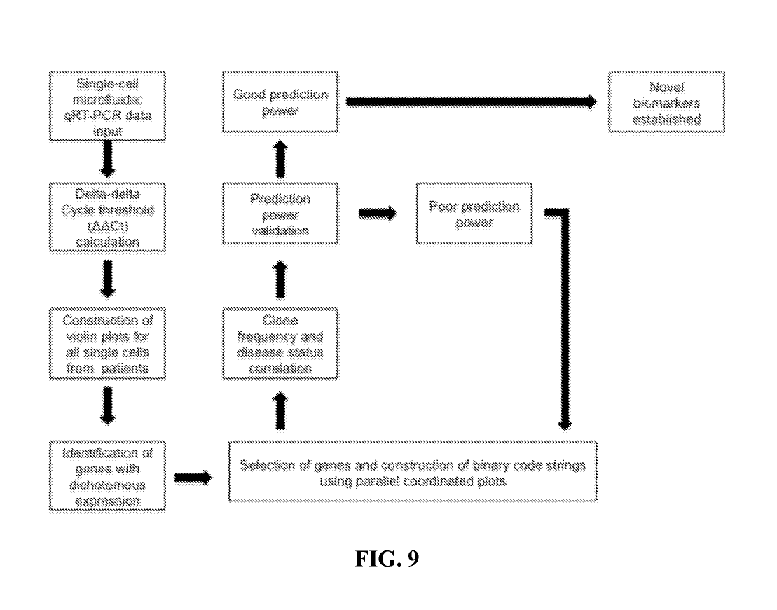

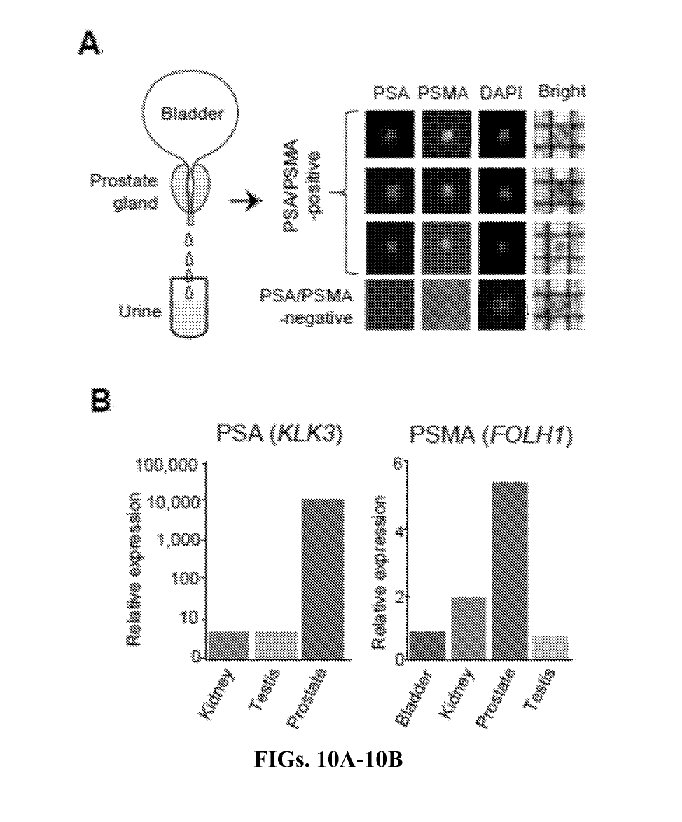

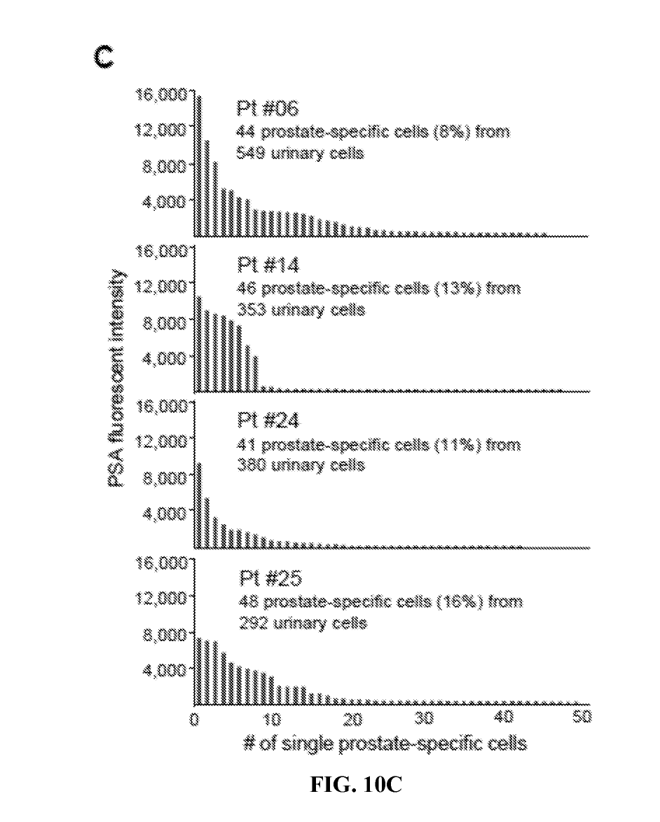

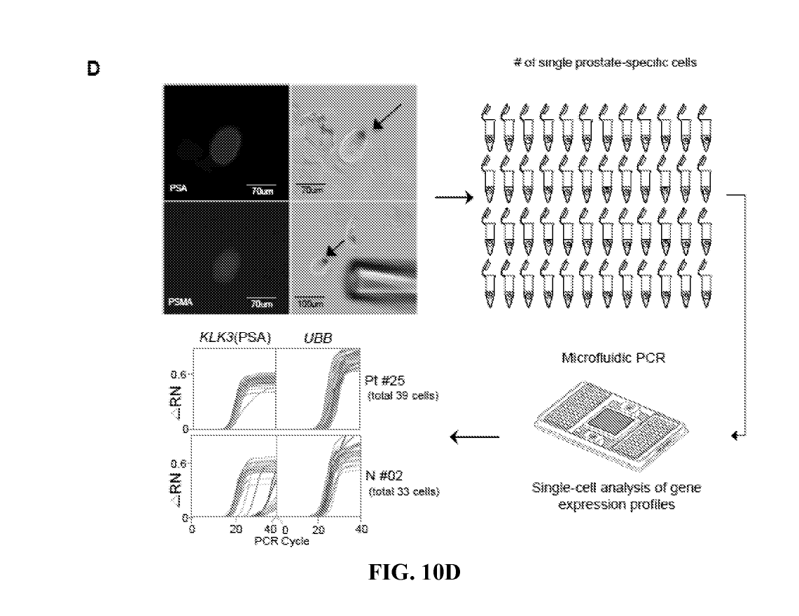

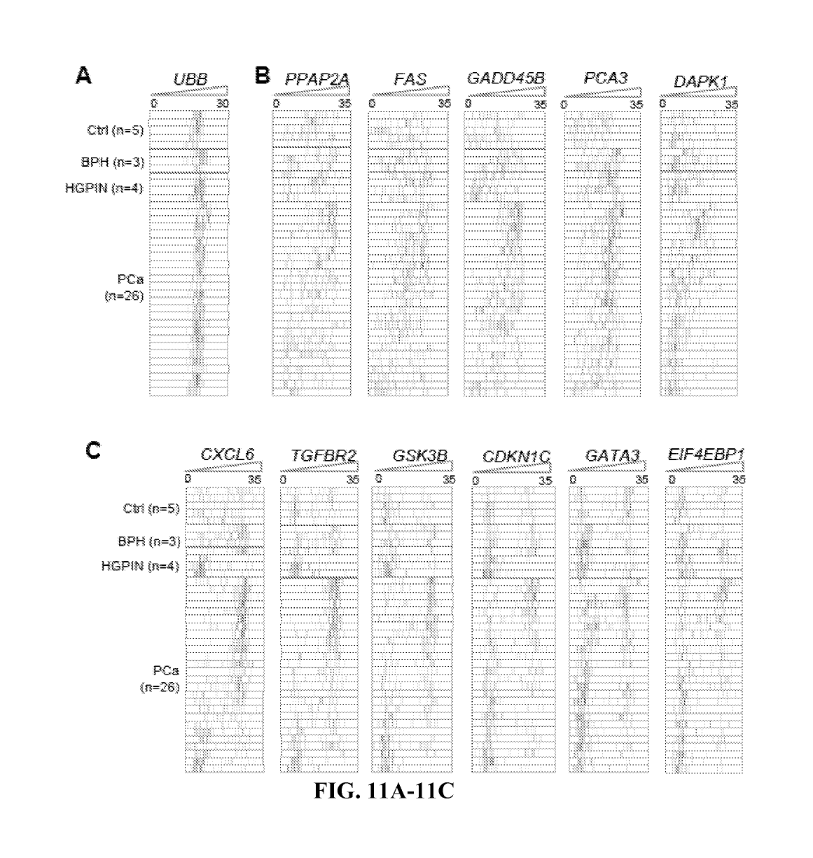

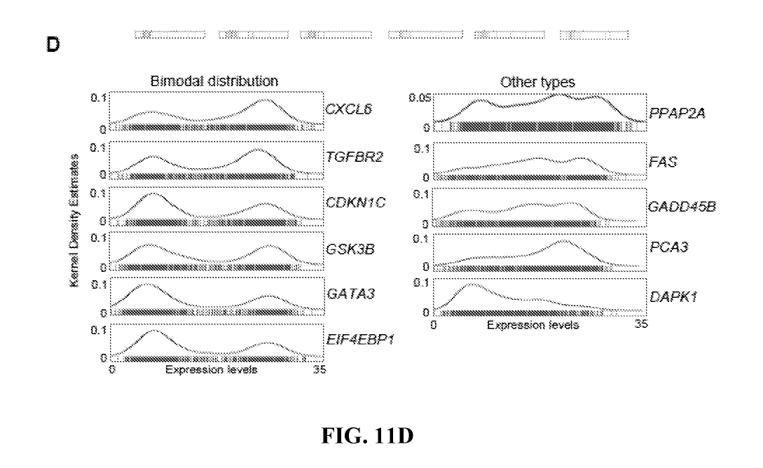

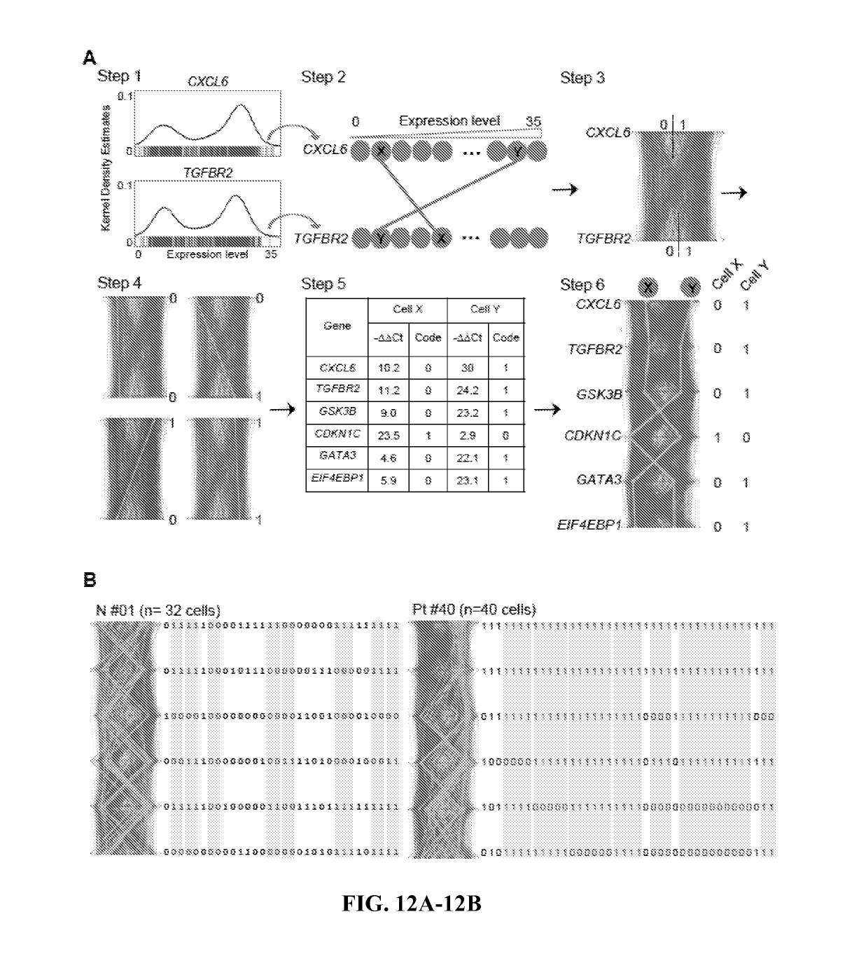

FIG. 9 illustrates an example of a method for establishing biomarkers for use in a single cell biomarker panel. In certain aspects of the method can be implemented using a computer system. A computer system can comprise instructions to receive, analyze, and determine if one or more biomarker or a set of biomarkers are effective in single cell biomarker profile assays. The computer system receives data from single cell PCR assay(s). The computer system calculates the delta-delta cycle threshold (ΔΔCt) for a candidate biomarker. The results of the ΔΔCt are transformed by the system into violin plots that include all single cell results from a given patient. The system identifies which biomarkers are dichotomously expressed. The system selects which biomarkers are dichotomously expressed and uses the selected biomarkers to construct binary code strings using parallel coordinated plots. The system assigns a binary code string associated with a biomarker panel to generate single cell biomarker profile that identifies a clone. The system assesses the correlation between clone frequency and disease status. The system analyzed the strength of the correlation using prediction power validation. If the clone frequency is a poor predictor then the system selects a new set of genes and constructs new binary code strings and then analyzes the new clones for correlation. If the clone is a good predictor then the system selects this code string as an established single cell biomarker panel.

Prostate Specific Antigen (PSA).

PSA is a peptidase of the kallikrein family and a differentiation antigen of the prostate. Alternate names include gamma-seminoprotein, kallikrein 3, seminogelase, seminin, and P-antigen.

Prostate Specific Membrane Antigen (PSMA).

PSMA, also known as Glutamate carboxypeptidase II, is a type 2 integral membrane glycoprotein found in prostate and a few other tissues. PSMA is expressed on tumor cells as a noncovalent homodimer.

Epithelial cell adhesion molecule (EpCAM).

EpCAM, also known as TACSTD1 (tumor-associated calcium signal transducer 1) and CD326 (cluster of differentiation 326), is a pan-epithelial differentiation antigen that is expressed on almost all carcinomas. It has been used as an immunotherapeutic target in the treatment of gastrointestinal, urological and other carcinomas. EpCAM is a carcinoma-associated antigen and is a member of a family that includes at least two type I membrane proteins. This antigen is expressed on most normal epithelial cells and gastrointestinal carcinomas and functions as a homotypic calcium-independent cell adhesion molecule.

Cytokeratins (CK7/8).

Cytokeratins constitute homology groups I and II. The nomenclature chosen in 1982 by Moll and Franke assign ranges from 1 to 8 for type I cytokeratins (neutral or alkaline) and from 9 to 12 for type II cytokeratins (acids). Cytokeratin 7 is a basic cytokeratin which is localized in most of glandular and transitional epithelial, but not in stratified squamous epitheliums. Cytokeratin 8 belongs to type B subfamily (alkaline) high molecular weight cytokeratins.

### V. CANCER TREATMENTS

In certain aspects, there may be provided methods for treating a subject determined to have cancer and with a predetermined expression profile of one or more biomarkers disclosed herein. In a further aspect, biomarkers and related systems, including biomarker expression profiles correlating to a particular DIGITAL BIOPSY™ binary code/clone as described herein, that can establish a prognosis of cancer patients can be used to identify patients who may benefit from conventional single or combined modality therapy. In the same way, the invention can identify those patients who do not benefit from such conventional single or combined modality therapy and can offer them alternative treatment(s).

In certain aspects of the present invention, conventional cancer therapy may be applied to a subject wherein the subject is identified or reported as having a good prognosis based on the assessment of the biomarkers as disclosed. On the other hand, at least an alternative cancer therapy may be prescribed, as used alone or in combination with conventional cancer therapy, if a poor prognosis is determined by the disclosed methods, systems, or kits.

Conventional cancer therapies include one or more selected from the group of chemical or radiation based treatments and surgery. Chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein tansferase inhibitors, transplatinum, 5-fluorouracil, vincristin, vinblastin and methotrexate, or any analog or derivative variant of the foregoing.

Radiation therapy causes DNA damage and has been used extensively, including what are commonly known as γ-rays, X-rays, and/or the directed delivery of radioisotopes to tumor cells or organs. Other forms of DNA damaging factors are also contemplated such as microwaves and UV-irradiation. Dosage ranges for X-rays range from daily doses of 50 to 200 roentgens for prolonged periods of time (3 to 4 wk), single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and/or a chemotherapeutic or radiotherapeutic agent are delivered to a target cell or are placed in direct juxtaposition with the target cell. In certain aspects both agents are delivered to a cell in a combined amount effective to kill the cell or prevent it from dividing.

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies. Curative surgery includes resection in which all or part, of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery).

Laser therapy is the use of high-intensity light to destroy tumor cells. Laser therapy affects the cells only in the treated area. Laser therapy may be used to destroy cancerous tissue and/or relieve a blockage when the cancer cannot be removed by surgery. The relief of a blockage can help to reduce symptoms.

Photodynamic therapy (PDT), a type of laser therapy, involves the use of drugs that are absorbed by cancer cells; when exposed to a special light the drugs become active and destroy the cancer cells.

Upon excision of part of all of cancerous cells, tissue, or tumor, a cavity may be formed in the body. Treatment may be accomplished by perfusion, direct injection or local application of the area with an additional anti-cancer therapy.

Alternative cancer therapy includes immunotherapy, gene therapy, hormonal therapy or a combination thereof. Subjects identified with poor prognosis using the present methods may not have favorable response to conventional treatment(s) alone and may be prescribed or administered one or more alternative cancer therapy per se or in combination with one or more conventional treatments.

### VI. COMPUTER IMPLEMENTATION

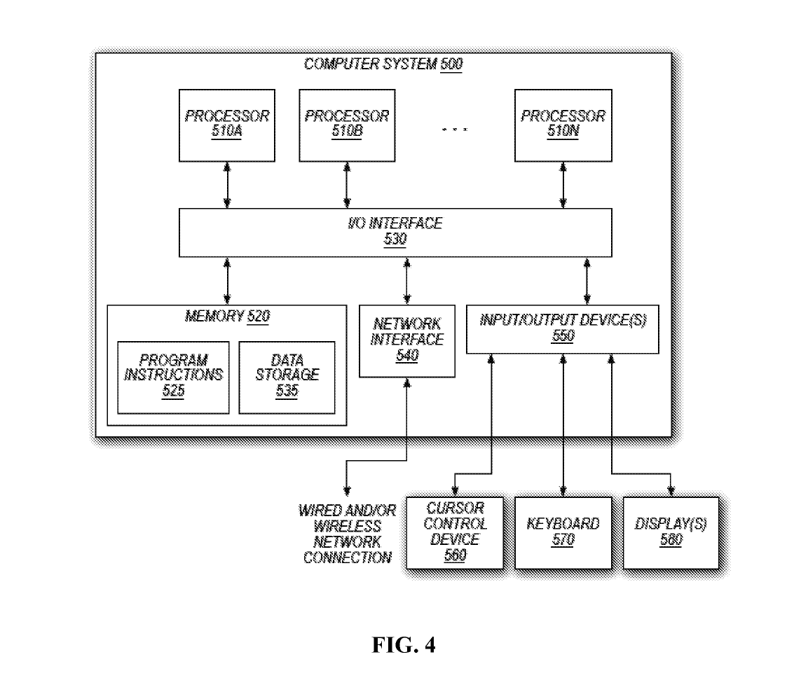

Embodiments of assays or methods described herein or the analysis thereof may be implemented or executed by one or more computer systems. One such computer system is illustrated in FIG. 4. In various embodiments, computer system may be a server, a mainframe computer system, a workstation, a network computer, a desktop computer, a laptop, or the like. For example, in some cases, the analysis described herein or the like may be implemented as a computer system. Moreover, one or more of servers or devices may include one or more computers or computing devices generally in the form of a computer system. In different embodiments these various computer systems may be configured to communicate with each other in any suitable way, such as, for example, via a network.

As illustrated, the computer system includes one or more processors 510 coupled to a system memory 520 via an input/output (I/O) interface 530. Computer system 500 further includes a network interface 540 coupled to I/O interface 530, and one or more input/output devices 550, such as cursor control device 560, keyboard 570, and display(s) 580. In some embodiments, a given entity (e.g., analysis of subjects for trypanosome infection and/or cardiomyopathy) may be implemented using a single instance of computer system 500, while in other embodiments multiple such systems, or multiple nodes making up computer system 500, may be configured to host different portions or instances of embodiments. For example, in an embodiment some elements may be implemented via one or more nodes of computer system 500 that are distinct from those nodes implementing other elements (e.g., a first computer system may implement an assessment of a hybrid latent variable assessment or system while another computer system may implement data gathering, scaling, classification etc.).

In various embodiments, computer system 500 may be a single-processor system including one processor 510, or a multi-processor system including two or more processors 510 (e.g., two, four, eight, or another suitable number). Processors 510 may be any processor capable of executing program instructions. For example, in various embodiments, processors 510 may be general-purpose or embedded processors implementing any of a variety of instruction set architectures (ISAs), such as the x86, POWERPC®, ARM®, SPARC®, or MIPS® ISAs, or any other suitable ISA. In multi-processor systems, each of processors 510 may commonly, but not necessarily, implement the same ISA. Also, in some embodiments, at least one processor 510 may be a graphics-processing unit (GPU) or other dedicated graphics-rendering device.

System memory 520 may be configured to store program instructions and/or data accessible by processor 510. In various embodiments, system memory 520 may be implemented using any suitable memory technology, such as static random access memory (SRAM), synchronous dynamic RAM (SDRAM), nonvolatile/Flash-type memory, or any other type of memory. As illustrated, program instructions and data implementing certain operations, such as, for example, those described herein, may be stored within system memory 520 as program instructions 525 and data storage 535, respectively. In other embodiments, program instructions and/or data may be received, sent or stored upon different types of computer-accessible media or on similar media separate from system memory 520 or computer system 500. Generally speaking, a computer-accessible medium may include any tangible storage media or memory media such as magnetic or optical media—e.g., disk or CD/DVD-ROM coupled to computer system 500 via I/O interface 530. Program instructions and data stored on a tangible computer-accessible medium in non-transitory form may further be transmitted by transmission media or signals such as electrical, electromagnetic, or digital signals, which may be conveyed via a communication medium such as a network and/or a wireless link, such as may be implemented via network interface 540.

In an embodiment, I/O interface 530 may be configured to coordinate I/O traffic between processor 510, system memory 520, and any peripheral devices in the device, including network interface 540 or other peripheral interfaces, such as input/output devices 550. In some embodiments, I/O interface 530 may perform any necessary protocol, timing or other data transformations to convert data signals from one component (e.g., system memory 520) into a format suitable for use by another component (e.g., processor 510). In some embodiments, I/O interface 530 may include support for devices attached through various types of peripheral buses, such as a variant of the Peripheral Component Interconnect (PCI) bus standard or the Universal Serial Bus (USB) standard, for example. In some embodiments, the function of I/O interface 530 may be split into two or more separate components, such as a north bridge and a south bridge, for example. In addition, in some embodiments some or all of the functionality of I/O interface 530, such as an interface to system memory 520, may be incorporated directly into processor 510.

Network interface 540 may be configured to allow data to be exchanged between computer system 500 and other devices attached to a network, such as electronic medical records systems, laboratory data reporting systems, health information exchange networks or other computer systems, or between nodes of computer system 500. In various embodiments, network interface 540 may support communication via wired or wireless general data networks, such as any suitable type of Ethernet network, for example; via telecommunications/telephony networks such as analog voice networks or digital fiber communications networks; via storage area networks such as Fiber Channel SANs, or via any other suitable type of network and/or protocol.

Input/output devices 550 may, in some embodiments, include one or more display terminals, keyboards, keypads, touch screens, scanning devices, voice or optical recognition devices, or any other devices suitable for entering or retrieving data by one or more computer system 500. Multiple input/output devices 550 may be present in computer system 500 or may be distributed on various nodes of computer system 500. In some embodiments, similar input/output devices may be separate from computer system 500 and may interact with one or more nodes of computer system 500 through a wired or wireless connection, such as over network interface 540.

As shown in FIG. 4, memory 520 may include program instructions 525, configured to implement certain embodiments described herein, and data storage 535, comprising various data accessible by program instructions 525. In an embodiment, program instructions 525 may include software elements of embodiments illustrated herein. For example, program instructions 525 may be implemented in various embodiments using any desired programming language, scripting language, or combination of programming languages and/or scripting languages (e.g., C, C++, C#, JAVA®, JAVASCRIPT®, PERL®, etc). Data storage 535 may include data that may be used in these embodiments. In other embodiments, other or different software elements and data may be included.

A person of ordinary skill in the art will appreciate that computer system 500 is merely illustrative and is not intended to limit the scope of the disclosure described herein. In particular, the computer system and devices may include any combination of hardware or software that can perform the indicated operations. In addition, the operations performed by the illustrated components may, in some embodiments, be performed by fewer components or distributed across additional components. Similarly, in other embodiments, the operations of some of the illustrated components may not be performed and/or other additional operations may be available. Accordingly, systems and methods described herein may be implemented or executed with other computer system configurations.

### VII. EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Single Cell Analysis of Post-DRE Urine Samples

About 20 ml or more post-digital rectal examination urine samples were centrifuged in 50 ml conical tubes at 400×g at 4° C. for 5 min. The urine supernatant is expirated until ˜2 ml of supernatant is left lest the urine cells in the pellets may be sucked out. The remaining supernatant is continuously removed using P1000 pipetteman. The pellets were subjected to two different processing.

For live cell processing, the pellets were subject washings and labeling with antibodies, for example:

(1) Cell pellets are suspended in 1 ml 1×PBS and transferred into a 1.5 ml centrifuge tube and spun in a bench microcentrifuge 400×g at 4° C. for 5 min. (2) 100 μl 0.05% trypsin is added at 37° C. for 10 min and spun in a bench microcentrifuge 400×g at 4° C. for 5 min. (3) Cell pellets are suspended in DMEM+5% FBS+P/S (1×) and labeled with α-PSA (1:100, Dako, #A0562), or α-hPSMA/FOLH1-APC (1:10, R&D system, #FAB4234A), incubate on ice for 15 min in a light proof environment (e.g., covered in aluminum foil). (4) The suspension is microcentrifuged 400×g at 4° C. for 5 min and washed with 1 ml DMEM+5% FBS+P/S (1×) twice to remove the primary (1°) antibodies. (5) Secondary (2°) Antibodies (anti-rabbit IgG-Cy3, 500× dilution) are applied in 200 μl DMEM+5% FBS+P/S (2%)+0.5 μg/ml DAPI at RT for 15 min. (6) The solution is microcentrifuged 400×g at 4° C. for 5 min and washed with 1 ml DMEM+5% FBS+P/S (1×) twice to remove the 2° antibodies. (7) The pellet is suspended in 20 μl DMEM+5% FBS+P/S (1×). (8) Immunostaining of cells is viewed under an Evos fl inverted microscope. (9) Cells are loaded onto DEPArray for single cell isolation and then BioMark molecular profiling using TBIIR and miRNA gene primer panel.

For fixed cells processing, the pellets were subject to washings, fixation, and antibody labeling as below:

(1) Urine sample (˜20 ml) centrifuged in a 50 ml conical tube at 400×g at 4° C. for 5 min. (2) Supernatant is removed gently without disturbing cell pellet. (3) Cell pellet is suspended in 1 ml 1×PBS and transferred into a 1.5 ml centrifuge tube and spun at 400×g at 4° C. for 5 min. (4) The supernatant is removed and 100 μl 0.05% trypsin is added and incubated at 37° C. for 10 min. (5) Solution is spun at 400×g at 4° C. for 5 min and the supernatant removed. (6) The cells are resuspended in 200 μl 1×PBS. (7) Cells are fixed with 2% formaldehyde for 20 min at room temperature. (8) Cells are spun a bench microcentrifuge for 20 seconds. (9) Cell pellets are suspended with 1 ml PBS+5% FBS+0.2% tween 20 and spun in a bench microcentrifuge for 20 seconds. (10) Cell pellets are suspended in 100 μl PBS+5% FBS+0.2% tween 20 and labeled with polyclonal rabbit α-PSA (1:100, DAKO, #A0562) and/or α-hPSMA/FOLH1-APC (1:10, R&D system, #FAB4234A) and incubated on ice for 15 min in the dark. (11) Suspension is spun in a bench microcentrifuge for 20 seconds and washed with 1 ml PBS+5% FBS+0.2% tween 20 to remove the 1° antibodies. (12) 2° Antibodies (anti-rabbit IgG-Cy3) are applied with 500× dilution in 100 μl PBS+5% FBS+0.2% tween 20+0.5 μg/ml DAPI (1:100 dilution) at RT for 15 min. (13) Sample is spun in a bench microcentrifuge for 20 seconds and wash with 500 μl SB115 buffer twice to remove the 2° antibodies. (14) Cells are suspended in 20 μl SB115 buffer. (15) Immunostaining of cells viewed under an Evos fl inverted microscope. (16) Cells are loaded onto DEPArray and subject to single cell analysis according to the protocol from Silicon Biosystems, Inc.

DEPArray Data Analysis.

About several dozens to three thousands urine cells are loaded unto DEPArray chips (cat# Silicon Biosystems, Inc) according to manufacturer's protocol. For live cells, the cells are suspended in DMEM+5% FBS+P/S (1×) or in SB115 buffer.

Analysis and Graphical Display in Prostate Cancer.

Single-cell analyses have revealed diverse patterns of gene expression in a cancer cell population (Meacham and Morrison, Nature 501, 328-337 (2013); Almendro et al., Annu Rev Pathol 8, 277-302 (2013)). The inventors describe a class of genes whose expression patterns can be reduced to binary codes at the single-cell level. Of 34 prostate cancer (PCa)-related genes examined in urinary cells originating from the prostate gland, six loci display the dichotomous characteristic that is coded as 0 for low and 1 for high expression, respectively. When arranging these genes in an order CXCL6-TGFBR2-GSK3B-CDKN1C-GATA3-EIF4EBP1, the inventors identify 64 (2*2*2*2*2*2) binary codes in 1220 single cells analyzed. Parallel coordinate plot (Swayne et al., Comput Stat Data An 43, 423-44 (2003)) is used to connect binary codes into a string (e.g., 111111, 101010, 010101, or 000000) for a single cell. Whereas these combinatorial codes are diverse in normal controls, unique code-strings are found in PCa patients. Furthermore, these code-strings represent different clonal populations of patient subgroups. High expression levels of tumor-promoting genes, including EPCAM and E2F1, are found in one subgroup, suggesting active clonal expansions of their cancer cells. Thus, the digital rendering of complex expression patterns enables identification of PCa cells in urine, providing a diagnostic adjunct to biopsy for cancer detection and risk assessment. This approach can also be used for clonal analysis of exfoliated cells for other diseases.

Epithelial cells exfoliated from the prostate gland are sometimes released into the urethra, thus appearing in urine (Ploussard and de la Taille, Nat Rev Urol 7, 101-09 (2010); Crawford et al., Diagnostic Performance of PCA3 to Detect Prostate Cancer in Men with Increased Prostate Specific Antigen: A Prospective Study of 1,962 Cases. J Urol (2012)). During the neoplastic process, a great number of abnormal prostate cells are exfoliated, providing a unique opportunity for cancer detection (Truong et al., J Urol 189, 422-29 (2013)). Previous analyses have confirmed cancer cells of the prostate origin in urine (Truong et al., J Urol 189, 422-29 (2013); Fujita et al. Hum Pathol 40, 924-33 (2009)), and prostate cancer antigen 3 (PCA3) is a urinary biomarker for PCa (Crawford et al. Diagnostic Performance of PCA3 to Detect Prostate Cancer in Men with Increased Prostate Specific Antigen: A Prospective Study of 1,962 Cases. J Urol (2012)). However, PCA3 has only moderate sensitivity and specificity for PCa detection (Crawford et al. Diagnostic Performance of PCA3 to Detect Prostate Cancer in Men with Increased Prostate Specific Antigen: A Prospective Study of 1,962 Cases. J Urol (2012); Whitman et al. J Urol 180, 1975-78 (2008)). Furthermore, PCa cells exfoliated in urine likely express diverse levels of PCA3, and the accurate measurement is frequently hampered when PCa cells are analyzed from a mixed urinary cell populations (Buganim et al. Cell 150, 1209-1222 (2012)).

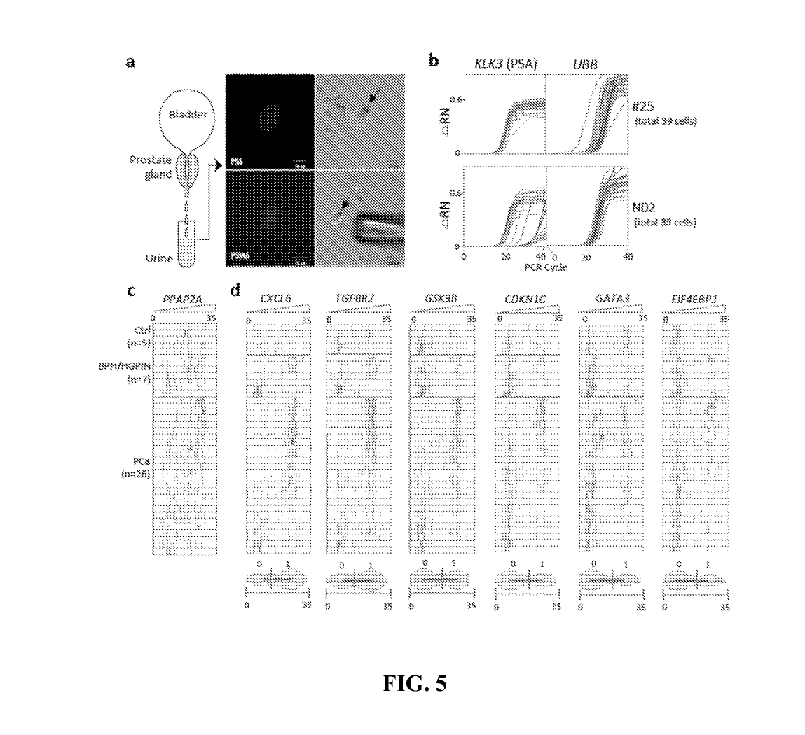

Motivated by the need to improve PCa detection, the inventors developed a method to analyze single-cell expression profiles (FIG. 5A). Exfoliated prostate cells in urine sediment were fluorescently stained with prostate-specific markers, PSA and PSMA (Ben Jemaa et al., J Exp Clin Cancer Res 29, 171 (2010)) and manually retrieved using a micromanipulator device. A total of 1283 exfoliated cells were collected from 33 patients undergoing prostate biopsy and from 5 healthy controls.

Single cells were subjected to microfluidic PCR analysis of 34 genes known to be aberrantly expressed in PCa (Cai et al. Cancer Cell 20, 457-71 (2011); Begley et al., Cytokine 43, 194-199 (2008)). A total of 1220 urinary prostate cells had robust expression values based on the cycle threshold (Cₜ) of amplification (FIG. 5B). Expression values of genes were normalized to that of a housekeeping gene, Ubiquitin B (UBB), which had stable expression values in prostate and other cell types (Popovici et al., BMC Bioinformatics 10, 42 (2009); Powell et al., PLoS One 7, e33788 (2012); Nikrad et al., Mol Cancer Ther 4, 443-49 (2005); Chen et al. Prostate 73, 813-26 (2013)). Expression levels of 28 genes, such as PPAP2A, varied extensively in single prostate cells (FIG. 5C). However, the remaining six genes exhibited a dichotomous expression pattern at the single-cell level (FIG. 5D). Violin plot analysis (Hintze and Nelson, The American Statistician 52, 181-84 (1998)) confirmed their bimodal expression distributions in prostate cells (FIG. 5D). A binary code system was used to digitize single-cell expression data with 0 as low and 1 as high expression, respectively. Binary codes of these genes were connected with a string for each cell in a parallel coordinate plot (PCP) (Swayne et al., Comput Stat Data An 43, 423-444 (2003)). Maps can be constructed that depict straight and crisscross strings between two genes (e.g., GATA3 and EIF4EBP1) for all cells analyzed. Code-strings (e.g., 00, 01, 10, and 00) can be shown for single cells. A third gene, e.g., CDKN1C, can added to produce eight possible code-strings 000, 100, 010, 001, 011, 101, 110, and 111. When arranging genes in order CXCL6-TGFBR2-GSK3B-CDKN1C-GATA3-EIF4EBP1, all 64 (2*2*2*2*2*2) possible code-strings can be identified in single cells analyzed. For the normal control N02, 19 code-strings were found in 32 single cells analyzed. Three code-strings-000000, 000010, and 100010 were repeatedly seen in 14 single cells, suggesting that code-string patterns are not randomly distributed in a population. Of note, the PCP of Patient #40 had a more homogenous pattern than that of N02, with only 13 code-strings being identified in 40 cells. Six of these code-strings-111011, 111101, 111110, 111111, 110100, and 111100 were frequently seen in the majority (80%) of single cells, suggesting the presence of specific clonal populations in this patient. In one study the inventors constructed 36 PCPs for clonal analysis of these prostate cells.

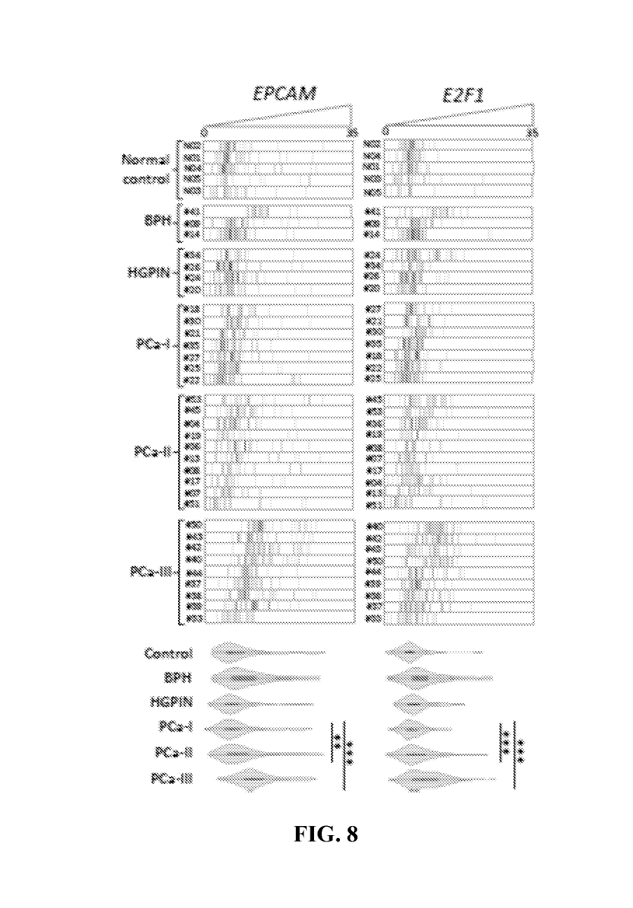

When categorizing code-strings into different classes, 21 code-strings were identified that distinguished different clonal populations of normal control, benign prostate hyperplasia (BPH), high-grade prostatic intraepithelial neoplasia (HGPIN), and PCa-I, -II, and -III subgroups. The Class A code-string (n=1) was frequently seen in normal control cells while Class B (n=4) and C (n=8) code-strings were commonly present in BPH and HGPIN groups, respectively. Interestingly, Class C code-strings were also found in clonal populations of PCa-I patients, confirming a clonal progression of malignancy from precursor HGPIN in this subgroup (Marusyk and Polyak, Science 339, 528-29 (2013)). Eight other code-strings—111111, 111110, 111101, 111011, 111010, 110010, and 101000 (Class D) were frequently present in PCa-II and -III subgroups. Compared to the former, PCa-III patients had large clonal populations (2-5 clones with ≧3 cells per clone) with Class D code-strings, suggesting active clonal expansions of their cancers. To confirm whether large Class D clones are associated with aggressive disease, single-cell expression data of the aforementioned PCa-related genes were analyzed in these patient subgroups (FIG. 8). Nineteen of 28 genes, including EPCAM and E2F1, were preferentially up-regulated in PCa-III cells compared with two other subgroups, PCa-I and -II (P<0.001). Indeed, EPCAM is known to be highly expressed in high-grade and advanced tumors (Ni et al., Cancer Metastasis Rev 31, 779-91 (2012)) while aberrant expression of E2F1 promotes the development of hormone-independent PCa (Davis et al., Cancer Res 66, 11897-906 (2006)). When examining patients' clinicopathological reports, six (#40, 37, 38, 39, 40, 42, and 44) of nine PCa-III patients had high-grade diseases and/or large tumor volume. However, three PCa-III patients (#33, 43, and 50) appeared to have low-risk PCa based on their biopsy results. As upgrading of low-risk PCa is seen in 30-50% of patients, further follow-up of these patients may confirm them to have aggressive tumors (Chun et al., Eur Urol 49, 820-26 (2006); Pinthus et al., J Urol 176, 979-984; discussion 984 (2006)). One PCa-II patient, #17, who also had aggressive PCa with bone metastasis, carried only a small Class D clone in his urinary prostate cells. Because his urine sample was collected at the time when the patient underwent a hormone ablation therapy, it is speculated that large aggressive clones were eliminated as a result of the therapy. Therefore, this single-cell technique can be offered not only as a diagnostic adjunct to prostate biopsy but also as a non-invasive monitoring of patients' response to treatment in the future.

PSA/PSMA-positive prostate cells were individually retrieved from urine sediment using a micromanipulator device. Cells lysed in reaction buffer were used for one-step CellsDirect™ RT-PCR analysis with the microfluidics system. Normalized values (−ΔΔCt) of genes were obtained for generating expression heat maps, violin graphs, and parallel coordinate plots of single cells. Connectivity paths of genes were converted into binary code-strings for clonal analysis.

Isolation of Urinary Single Cells of the Prostate Origin.

Patient consent for the urine collection was carried out according to IRB protocol approved at the University of Texas Health Science Center San Antonio (UTHSCSA). Urine samples (˜25 mL) collected in a container were transferred onto a 50 ml conical tube and kept on ice for immediate processing. Urinary cellular components were precipitated at 400×g at 4° C. for 5 min. The supernatant was removed gently without disturbing cell pellets. Cell pellets were suspended with 1 mL 1×PBS and transferred onto a 1.5 mL low-retention centrifuge tube and spun down at 400×g for 5 min at 4° C. The wash and centrifugation were repeated. Cell pellets were suspended in 100 mL 0.05% trypsin to dissociate cell aggregates at 37° C. for 10 min and then was neutralized with 500 μl DMEM+5% FBS supplemented with penicillin/streptomycin (P/S), 100 unit/ml and 100 μg/ml, respectively, and centrifuged at 4° C. at 400×g for 5 min. After the supernatant was removed, cell pellets were suspended in 100 μl DMEM+5% FBS+P/S and labeled with polyclonal rabbit α-PSA (v:v=1:100, Dako, #A0562), mouse α-hPSMA(FOLH1)-APC (v:v=1:10, R&D system, #FAB4234A) on ice for 15 min with light proof. The cells were microcentrifuged at 400×g at 4° C. for 5 min and washed with 1 mL DMEM+5% FBS+P/S twice to remove 1° antibodies. A secondary antibody (α-rabbit IgG-Cy3) was applied in a 500-fold dilution with 200 μl DMEM+5% FBS+P/S+0.5 ug/ml DAPI at RT for 15 min on ice. The cells were centrifuged at 400×g for 5 min at 4° C. and subsequently washed with 1 ml DMEM+5% FBS+P/S twice to remove the secondary antibody. The cells were resuspended in 20 μl DMEM+5% FBS+P/S (1×) and examined for immunostaining under an Evos fl inverted microscope. Single PSA/PSMA+ prostate cells were isolated using a combined micromanipulator-microinjector system (CM2S) (Chen et al., Prostate 73, 813-26 (2013)) and lysed in 4 mL 2× reaction buffer (CellDirect™ one step qRT-PCR kit, Invitrogen, Inc) and frozen at −80° C. immediately until further use.

Single-Cell Microfluidic PCR.

Single-cell microfluidics-based RT-PCR analysis was carried out using CellsDirect™ one-step qRT-PCR kit (Invitrogen, Carlsbad, Calif.) with modifications and a microfluidics device, BioMark HD MX/HX system (Fluidigm, South San Francisco, Calif.) (Chen et al., Prostate 73, 813-26 (2013)). Three μl of lysate (˜1/3) of a urinary single cell was subject to PCR amplification using a panel of 34 prostate cancer-related genes and a control gene, Ubiquitin B (UBB). To reduce contamination, genomic DNA from the lysate was degraded in a 18-μl reaction using DNase I (5 units) with 1×DNase I buffer at RT for 5 min. PCR primers of selected genes for expression profiling were selected from the PrimerBank database. A primer mixture (500 nM) for each panel was prepared in TE buffer by pooling all the primers of each panel.

Reverse transcription and pre-amplification were carried out in a 10 μl reaction with 3 μl single-cell total RNA in 1× CellDirect™ reaction mix, 2% SuperScript III RT platinum Taq mix and 50 nM primer mix. RT was performed at 50° C. for 15 sec and inactivated at 95° C. for 2 min. Followed are 20 thermal cycles of pre-amplification: 95° C. (15 sec) and 60° C. (4 min). Excessive primers in pre-amplification were removed by 18 units of Exonuclease I (Exo I) at 37° C. for 30 min. Pre-amplified products were diluted 1:1 with H₂O before PCR using a BioMark microfluidic instrument.

For PCR amplification, the pre-amplified products were premixed with 1×SsoFast EvaGreen supermix with low ROX (Bio-Rad, Hercules, Calif.) and 1×DNA binding dye sample loading reagent (Fluidigm). Sample and primer pre-mixtures were loaded unto 48×48 array chips according to manufacturer's protocol (cat # BMK-M-48.48, Fluidigm). Pre-amplification from about 200 pg universal mRNA and H₂O are used for positive and negative controls on each 48×48 Dynamic Array.

Cell Culture, siRNA Transfection and RT-qPCR.

LNCaP and PC3 cells routinely grown in the laboratory were transfected with SMARTpool siRNAs specifically targeting TGFBR2 and GSK3B, respectively, according to the manufacturer protocol (ThermoFisher). Unspecific scrambled siRNA was used as a negative control. Total RNA were prepared from transfected and control cells or from cells treated with R1881 (1 nM), a synthetic androgen agonist. Reverse transcription was performed using the SuperScript III kit (Invitrogen) on a Veriti 96-well Thermocycler (Applied Biosystems). Relative expression values of genes were shown as the average fold changes (2−ΔΔCt) from three individual experiments using UBB as an internal control and the untreated expression designated as 1.

Cell Proliferation, Migration, and Invasion Assays.

Cells were seeded in triplicate wells of a 96-well plate, and every three hours four independent images were taken per well and quantified for confluence percentage using the IncuCyte ZOOM system. To eliminate variations of cell size and seeding densities, relative cell densities at all time points' values were normalized to the initial time point's average confluence.

Cell monolayers were seeded to optimized confluent levels in a 96-well ImageLock Plate (Essen BioSciences). The 96-well WoundMaker created equal scratches in every well simultaneously for cell migration and invasion analysis (Essen BioSciences). For invasion assays, BD Matrigel™ Basement Membrane Matrix (BD Biosciences) was used for coating the ImageLock plates, creating a thick layer above the scratched monolayer, 0.1 and 4.0 mg/ml respectively. The IncuCyte ZOOM system's scan mode set to scratch wound and wide mode and captured 1 image every 3 hours. Migration and invasion were analyzed using the relative wound density percent (RWD %) metric.

Quantitation of Gene Expression in Single Cells.

Expression levels of 35 genes, obtained as threshold cycle (Cₜ) values, were normalized to that of the control reference gene UBB and displayed as −ΔΔCₜ values (Livak and Schmittgen, Methods 25, 402-08 (2001)). The UBB gene was used as a control because its mRNA was found to be highly stable in single prostate cells in our previous microfluidics-based PCR assays (Chen et al., Prostate 73, 813-26 (2013)). The inventors selected cells that expressed UBB at a threshold of Cₜ≦30 after pre-amplification, assuming that these cells expressing robust expression of UBB are less likely to contain degraded RNA. The −ΔΔCₜ values ranged from the lowest expression level of 0 to the highest expression level of 35, which were used to construct expression heatmaps.

Violin Plot Analysis.

A violin expression plot, which combines a box plot and a rotated kernel density plot (Hintze and Nelson, The American Statistician 52, 181-184 (1998)), were constructed for each gene to determine clonal distributions of gene expression in a given population of prostate cells. The density trace is plotted symmetrically to the left and the right the vertical box plot, and there is no difference in these density traces other than the direction in which they extend. Median expression levels of these genes from urinary single cells isolated in (1) normal controls and patients diagnosed with (2) benign prostate hyperplasia (BPH), (3) prostatic intraepithelial neoplasia (PIN) and (4) prostate cancer were analyzed using one-way ANOVA and unpaired Student's t test using R. A P value of <0.05 is considered as statistically significant.

Kernel Density Plot and Parallel Coordinate Plot Analysis.

Expression patterns of selected genes in single exfoliated prostate cells of a patient were displayed by Kernel density plots using the ‘density’ function in R (Venables and Ripley, 2002). The density estimation displays the distribution of the expression levels with a smoothing parameter that accounted for the density of cells inside a defined interval of expression levels. Expression patterns were visualized in parallel coordinates plots using the software of GGobi data visualization system (Swayne et al., 2003). Each parallel coordinate plot was composed of points and lines. The points, referring to cells, were arranged from the lowest expression level in the left to the highest in the right end. The lines linked to these points displayed expression connectivity among the genes. Expression paths of selected cells for each patient were highlighted.

In Silico Analysis of Gene Expression.

Gene expression (RNA-seq) data of adjacent normal (n=37) and primary PCa (n=140) used for this study were obtained from The Cancer Genome Atlas (TCGA). In order to display the expression level of selected genes in the same heat map, TCGA data were adjusted using Normalize Genes/Rows function in the software of MultipleExperiment Viewer 4.8. This process standardized gene expression values using the mean and the standard deviation of the row of the matrix to which the gene belongs. The difference between Prostate samples and Normal samples was further compared by Student's t-test using Prism 6 (GraphPad Software, La Jolla, Calif.). A P value of <0.05 is considered as statistically significant.

Digitizing Expression Levels into Binary Codes.

Gene expression levels were binarized into 1 or 0 for the high expression group and low expression group respectively based on the bimodal distribution observed in Kernel density plots. The values used to define high and low groups for each gene were cutting points that can best separate two conjunct normal distributions in the plots. The value is 14.5 (−Ct) for CXCL6, 15 TGFBR2, 17 GSK3B, 17 CDKN1C, 17.5 GATA3, and 18 EIF4EBP1.

Statistical Resampling Strategy.

A two-layer resampling frame was developed to sample cells from the 832 existing cells to construct simulation groups for identify unique clonally-associated numerical patterns (CANPs). In the first layer, one group from normal controls, benign prostate hyperplasia, low-risk group prostate cancer (including high-grade prostatic intraepithelial neoplasia), and high-risk group prostate cancer was randomly selected. This step could avoid the bias of the unequal percentages of cells in different groups. In the following layer, a cell was randomly drawn from the previously selected group. The cell being drawn was put back into the pool for resampling. The inventors repeated this two-layer resampling process until enough cells were being drawn for a simulation group.

Polar Plot Analysis.

Polar plots displayed proportions of CANPs in simulation and observed groups of cells. The average (μ) proportions of CABCs from 2000 simulation groups were portrayed as solid lines in each polar plots, and shadow spans covered 1.7 times of standard deviations (S.D.) from the average (Normal, BPH, low-risk group, and high-risk groups). The asterisk denotes the observed proportions of code-strings.

Euclidean Distance Analysis and Experimental Distribution.

Euclidean distance was used to measure the similarity of code components in any of two observed groups (e.g., high-risk prostate cancer versus low-risk prostate cancer groups). The equation of the Euclidean distance is as follows:

D=\sqrt{Σᵢ(pᵢ−qᵢ)²}

Where p and q stand for two groups, and i represents one of 38 distinct CANPs. The Euclidean distance calculated the differences of the proportion of a CANP (i) in cells between two groups (p and q). An experimental distribution of Euclidean distance was constructed from 5000 pairs of simulation groups, which were generated by the two-layer resampling frame. This experimental distribution displayed the frequency of the Euclidean distance calculated based on the 5000 times of the simulation.

Statistical Analysis.

Statistical analysis was done using GraphPad Prism 5.04 software. Two-tailed Student's t-test was applied to analyze differences in relative expression levels between control groups and knockdown groups, and also in the percent of wound density for migration and invasion assays. Two-way ANOVA was used to compare differences in cell line groups with and without treatments. Kernel density estimates was examined using the ‘density’ function in R software (Venables and Ripley, 2002). All experiments in migration and invasion for cell cultures were performed independently at least three times and at least triplicate each time. All data were presented as mean±SD. In all cases, p<0.05 were considered statistically significant. For quantification of gene expression in single cells, kernel density plots, parallel coordinate plots, digitizing binary codes, the resampling strategy, polar plot analysis, and Euclidean distance analysis, please see Supplemental Experimental Procedures.

### Example 2

### Bimodality of Single-Cell Gene Expression Defines Subpopulations of Exfoliated Prostate Cells in Urine for Risk Evaluation of Prostate Cancer

Single Exfoliated Prostate Cells Display Wide-Ranging PSA Values in Urine Sediment from PCa Patients.

Urine contains a collection of waste products, including epithelial cells shed from kidney tubules, ureteral and bladder lining, and lower urinary tract (Vrooman and Witjes, 2008; Kim et al., 2009). Normal prostate ducts also exfoliate epithelial cells through a renewal process and release them into the urethra, thereafter appearing in urine (Ploussard and de la Taille, 2010; Crawford et al., 2012). During the neoplastic process, many abnormal prostate cells are exfoliated from the prostate due to the weakening of the cell-to-cell and cell-to-matrix contacts (Truong, 2013). To survey the extent of this prostate-specific exfoliation in urine, urinary cells were pre-stained with two prostate-specific markers, PSA and prostate specific membrane antigen (PSMA), and used the DEPArray System™ to array individual cells in a dielectrophoresis chip. A wide range of DAPI-positive urinary cells (average 393 cells) were counted in ˜10 ml urine from four PCa patients (FIG. 1C). Consistent with previous findings, 8-16% (n=41-48) of these cells were of prostatic origin. While the majority of exfoliated prostate cells exhibited substantial differences in PSA values in three patients (#06, 24, and 25), Pt #14 likely exhibited two distinct populations of high- and low-PSA-containing cells (FIG. 1C). The initial measurement of single-cell PSA fluorescence intensity provides a basis for further evaluation of expression heterogeneity in exfoliated prostate cell populations of PCa patients.

Single Exfoliated Prostate Cells Exhibit a Bimodal Pattern of Gene Expression in a Subset of PCa-Promoting Loci.

To assess clonal patterns of exfoliated prostate cells, expression analysis of 34 PCa-promoting genes associated with androgen-receptor or TGF-b signaling pathways were conducted in a total of 1329 single PSA/PSMA-positive cells cherry-picked with a micromanipulator device and aspirated into PCR tubes (FIG. 1D; Table 1). These cells were isolated from urine samples of patients with benign prostate hyperplasia (BPH, n=3), high-grade prostatic intraepithelial neoplasia (HGPIN, n=4), and PCa (n=26; Table 2). A total of 190 single prostate cells were also isolated from urine sediments of five normal controls undergoing vasectomy.

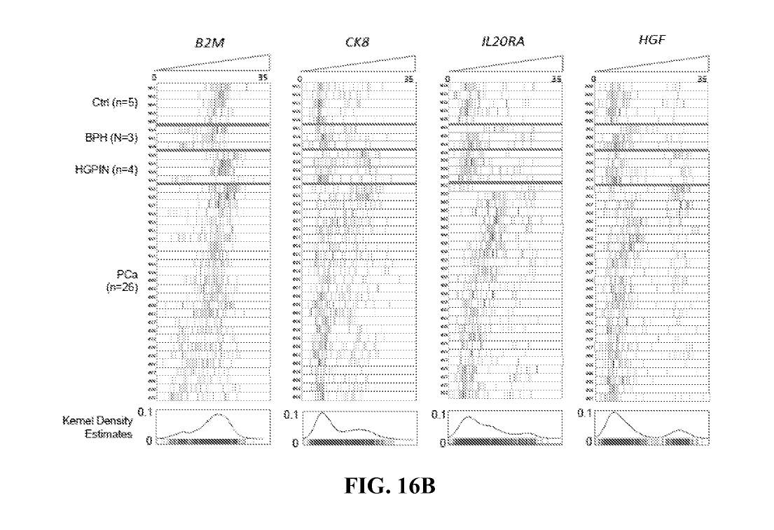

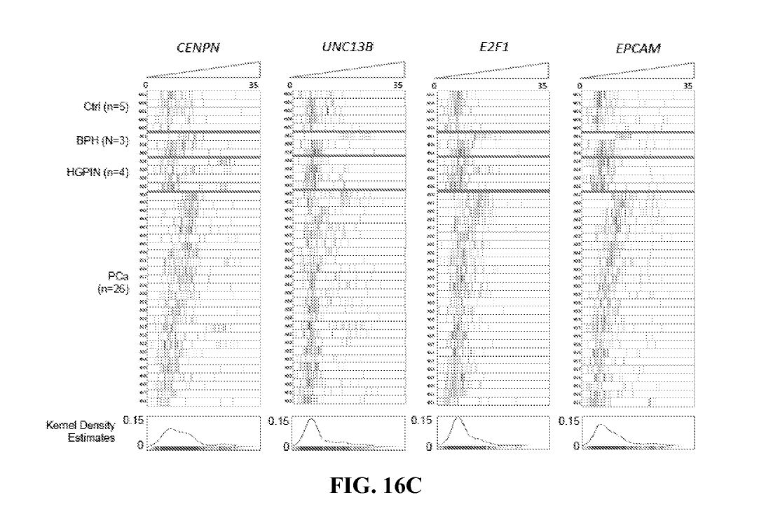

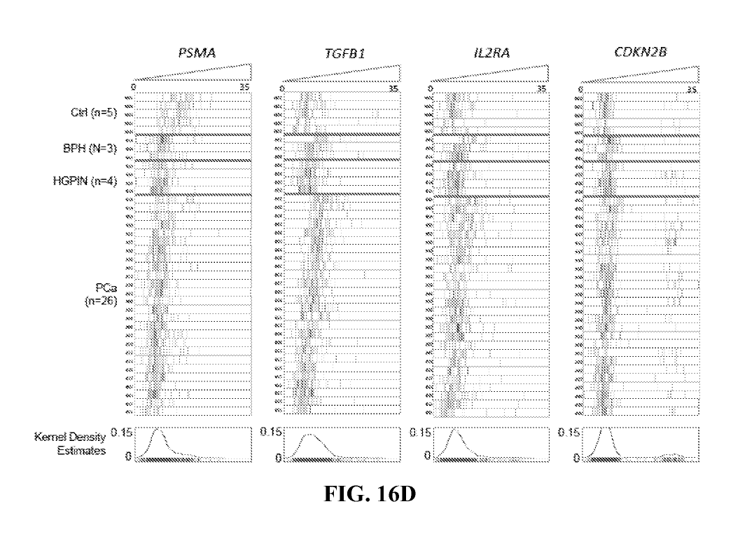

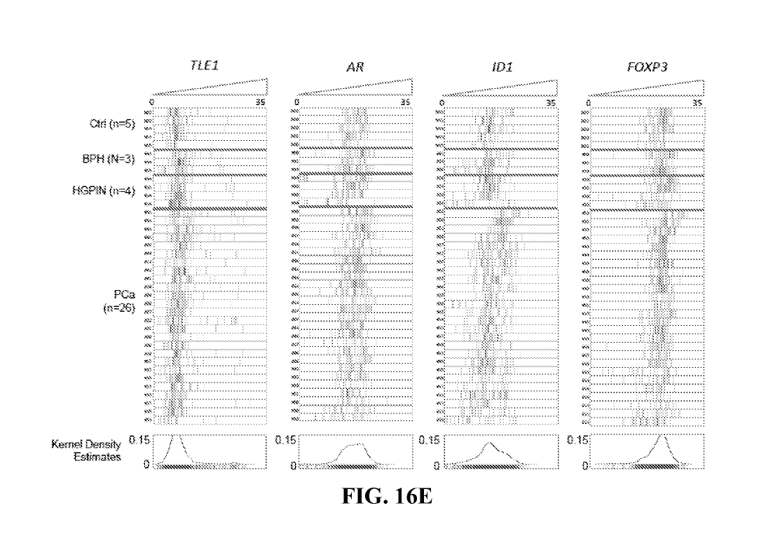

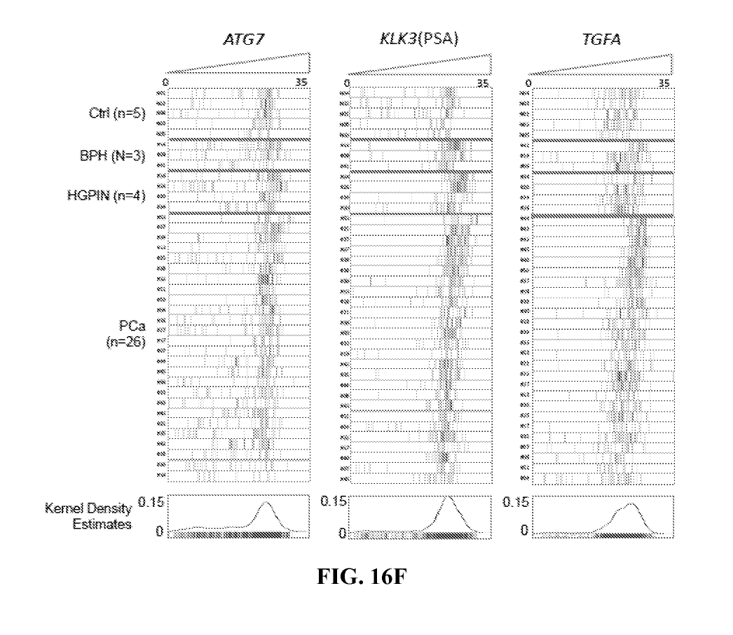

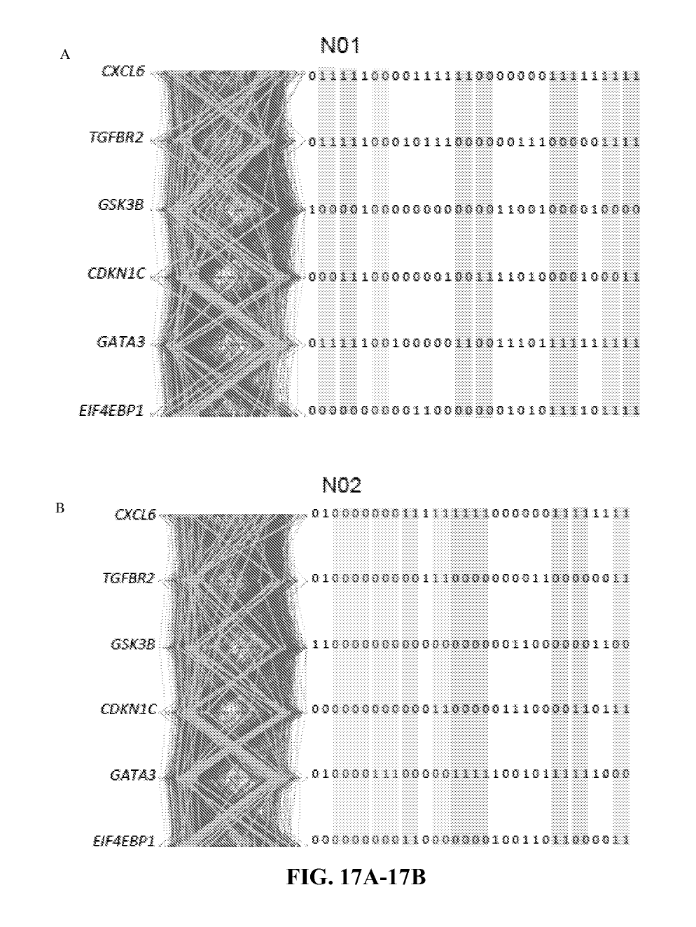

Exfoliated prostate cells (on average 35 single cells per sample) isolated from normal controls and patients were subjected to microfluidic RT-PCR analysis, and 1220 (92%) of 1329 cells exhibited robust expression values based on the cycle threshold (Ct) of amplification (see representative examples in FIG. 1D, lower-left). Relative expression values (−ΔΔCt) of a gene in these cells were first normalized to that of Ubiquitin B (UBB), a housekeeping gene that had stable Ct values in prostate and other cell types, and then normalized to the lowest expression of the gene (FIG. 2A). Expression of 28 PCa-promoting genes varied extensively in single prostate cells (FIG. 2B; FIG. 16). Interestingly, the remaining six genes—CXCL6, TGFBR2, GSK3B, CDKN1C, GATA3, and EIF4EBP1 frequently exhibited a bimodal expression pattern of high and low-expressing cell populations in BPH, HGPIN, and PCa groups (FIG. 2C). These genes are upstream modulators/effectors and downstream targets of TGF-b that has a tumor suppressive effect on cancer development, but may act as a promoter for invasion and metastasis in advanced cancer (Hannigan et al., (2010). J Clin Invest 120, 2842-57; Lebrun, (2012) ISBN Mol Biol, 1-28). Kernel density estimation confirmed bimodal expression distributions of these six genes in exfoliated prostate cell populations (FIG. 2D, left). These bimodally expressed genes have the potential to serve as biomarkers to stratify subpopulations of exfoliated prostate cells in PCa patients.

TABLE 1
Thirty-four genes and their functions related to prostate cancer.

| Gene symbol | Name | PCa-related function |
|---|---|---|
| AR | Androgen receptor, | Growth and progression (Sampson et al., 2013) |
| ATG7 | Autophagy related 7 | Cytoprotective autophagy (Zhu et al., 2010) |
| B2M | Beta-2-microglubulin | Upregulation for cell survival (Gross et al., 2007; Mink et |
| | | al., 2010) |
| BIK | BCL2-Interacting killer (apoptosis-inducing) | Susceptibility gene (Kim et al., 2010; Schumacher et al., |
| | | 2011), upregulation (Nikrad et al., 2005) |
| CDKN1A | Cyclin-dependent kinase inhibitor 1A | Downregulated in tumor but upregulated in androgen- |
| | (p21, Cip1) | independency (Fizazi et al., 2002; Romics et al., 2008) |
| CDKN1C | Cyclin-dependent kinase inhibitor 1C | Cell growth inhibition and senescence (Jin et al., 2008) |
| | (p57, Kip2) | |
| CDKN2B | Cyclin-dependent kinase inhibitor 2B (p15, | Cell growth inhibition (Guo and Kyprtanou, 1998) in high- |
| | INK4B) | grade carcinomas (Sherwood et al., 1991; Zhang et al., |
| | | 2006) |
| CENPN | Centromere protein N | |
| KRT8 | Keratin B | Increased expression in metastasis (Hofmann et al., 2003) |
| CXCL0 | Chemokine (C-X-C motif) ligand 6 | Upregulated in inflammation (Begley et al., 2008) |
| DAPK1 | Death-associated protein kinase 1 | Hypermethlated promoter (Wang et al., 2004) |
| E2F1 | E2F transcription factor 1 | Elevated in metastasis (Davis et al., 2006) and |
| | | apoptosis(Libertini et al., 2006) |
| EIF4EBP1 | Eukaryotic translation initiation factor 4E | Overexpression (Kremer et al., 2005) and reduced patient |
| | binding protein 1 | survival (Graff et al., 2009) |
| EPCAM | Epithelial cell adhesion molecule | Overexpression (Wert et al., 2006) |
| FAS | TNF receptor superfamily, member 6 | Induction of apoptosis (Srikanth et al., 1999) |
| FOXP3 | Forkhead box P3 | Tumor suppressor gene (Li et al., 2011; Valdman et al., |
| | | 2010) |
| GADD45B | Growth arrest and DNA-damage- | Cell death protein (Yang et al., 2006) |
| | inducible, beta | |
| GATA3 | GATA binding protein 3 | Tumor suppressor gene (Nguyen et al., 2013) in PSA |
| | | expression (Perez-Stable et al., 2000; Wang et al., 2004) |
| GSK3B | Gycogen synthase kinase 3 beta | Suppression of cell growth (Wang et al., 2004) |
| HGF | Hepatocyte growth factor | Tumor growth (Nishimura et al., 2008) and invasion (Hall et |
| | | al., 2004) |
| ID1 | Inhibitor of DNA binding 1, | Cell survival and proliferation (Schmidt et al., 2010; Xu et |
| | | al., 2009) |
| ID3 | Inhibitor of DNA binding 3, | Proliferation (Asirvatham et al., 2007) |
| IL20RA | Interleukin 2D receptor, alpha | |
| IL2RA | Interleukin 2 receptor, alpha | Antitumor (Wu and Xu, 2010) |
| NKX3-1 | NK3 homeobox 1 | Frequent deletion (He et al., 1997) and suppression of Myc |
| | | (Anderson et al., 2012) |
| PCA3 | Prostate cancer antigen 3 | Upregulation (Anherst et al., 2008) |
| PPAP2A | Phosphatidic acid phosphatase type 2A | Downregulation (Porkka and Visakorpi, 2001) |
| KLK3 (PSA) | Kallkrein-related peptidase 3 | Upregulation (Grossitatus et al., 2001) |
| FOLH1 | Folate hydrolase 1 | Suppressing invasiveness (Ghohs et al., 2005; Silver et al., |
| (PSMA) | | 1997) |
| TGFA | Transforming growth factor, alpha | Upregulation (Ching et al., 1993) |
| TGFB1 | Transforming growth factor, beta 1 | Apoptosis (Zhu et al., 2008) and poor prognosis (Levy and |
| | | Hill, 2005; Schroten et al., 2012) |
| TGFBR2 | Transforming growth factor, beta receptor II | Progression (Levy and Hill, 2006; Li et al., 2008) |
| TLE1 | Transducin-like enhancer of split 1 | Upregulation (Nakaya et al., 2007) |
| UNC13B | Unc-13 homolog B | Apoptotic gene (Song et al., 1999) |

TABLE 2
Patient Information. BPH: Benign prostate hyperplasia; HGPIN: high-grade prostatic
intraephithelial neoplasia; PCa: Prostate cancer; GS: Gleason Score on a histological scale of 1-5
for a dominant and a secondary score; and PSA: prostate-specific antigen.

| | | | # of cells | |
|---|---|---|---|---|
| Grouping | Patient | Age | picked (analyzed) | Biopsy results |
| Normal | N01 | n/a | 32(32) | |
| | N02 | n/a | 33(33) | |
| | N03 | n/a | 47(24) | |
| | N04 | n/a | 33(32) | |
| | N05 | n/a | 45(15) | |
| BPH | 9 | 69 | 35(35) | PSA 5.9 ng/ml; biopsy negative for cancer. |
| | 14 | 59 | 56(48) | PSA 5.0 ng/ml; biopsy negative for cancer. |
| | 41 | 69 | 24(24) | PSA unavailable; biopsy negative for cancer. |
| HGPIN | 20 | 62 | 35(35) | PSA 6.3 ng/ml; biopsy 2 cores with precursor lesions. |
| | 24 | 61 | 48(45) | PSA 3.7 ng/ml; fell 3.7 ng/ml; biopsy, 2 cores, focal lesions. |
| | 26 | 65 | 44(43) | PSA 4.9 ng/ml; biopsy, precursor lesions. |
| | 34 | 55 | 24(22) | PSA 7.9 ng/ml; biopsy, 1 core, precursor lesions. |
| Low risk | 7 | 45 | 26(26) | PSA 1.5 ng/ml; biopsy, 5/12 cores, GS 3 + 3 ~15% tumor |
| PCa | 22 | 53 | 50(49) | PSA 5.2 ng/ml; biopsy, GS 3 + 3 <1 mm. |
| | 27 | 54 | 40(40) | PSA 1.9 ng/ml; biopsy, GS 3 + 3 <1 mm. |
| | 33 | 49 | 30(30) | PSA 2.3 ng/ml; biopsy, GS 3 + 3, 1 mm; and repeat biopsy, |
| | | | | GS 3 + 3 <1 mm. |
| | 35 | 58 | 40(40) | PSA 2.8 ng/ml; biopsy, 2 cores, GS 3 + 4 <1 mm and |
| | | | | HGPIN. |
| | 43 | 69 | 32(32) | PSA 1.5 ng/ml; biopsy, 1 core, GS 3 + 4 3% tumor and 1 |
| | | | | core, GS 3 + 3, 3% tumor; and repeat biopsy negative. |
| | 50 | 57 | 40(40) | PSA 6.1 ng/ml; biopsy, 2 cores, GS 3 + 3, 5% tumor. |
| | 51 | 65 | 21(21) | PSA 4.1 ng/ml; biopsy, 1 core, GS 3 + 3, 1% tumor. |
| | 53 | 76 | 24(24) | PSA 5.5 ng/ml; biopsy, 3 cores, GS 3 + 3 <5, 20, and |
| | | | | 40% tumor. |
| High risk | 4 | 64 | 36(36) | PSA 29.1 ng/ml; biopsy, 1/11 cores, GS 3 + 4 30% tumor, and |
| PCa | | | | 10/11 cores, GS 4 + 3, 10-95% tumor. |
| | 8 | 59 | 23(22) | PSA 1.6 ng/ml; initial biopsy, GS 3 + 3; repeat biopsy, |
| | | | | GS 3 + 4, 35% tumor, 2 cores. |
| | 13 | 67 | 23(23) | PSA 7.9 ng/ml; biopsy, 4 cores, GS 3 + 3; 5-35% tumor, and |
| | | | | 1 core, GS 3 + 4, 20%. |
| | 18 | 69 | 26(26) | PSA 2.9 ng/ml; initial biopsy, GS 3 + 3; repeat biopsy, |
| | | | | GS 3 + 4, 35% tumor. |
| | 21 | 69 | 32(31) | PSA 5.9 ng/ml; initial, GS 3 + 3; repeat biopsy, 5 cores, |
| | | | | GS 3 + 3, 20% tumor, and one core, GS 3 + 4. |
| | 25 | 65 | 40(39) | PSA 5.77 ng/ml; biopsy, 3 cores, GS 4 + 4. |
| | 30 | 50 | 31(29) | PSA 9.5 ng/ml; biopsy, 1 core, GS 4 + 4; 1 core, GS 4 + 3; |
| | | | | 4 cores, GS 3 + 4; and 1 core, GS 3 + 3. |
| | 36 | 59 | 62(30) | PSA 7.5 ng/ml; biopsy, 2/12 cores, right lobe, GS 3 + 3; |
| | | | | and 6/12 cores, left lobe GS 3 + 4. |
| | 37 | 65 | 40(40) | PSA 4.1 ng/ml; biopsy, GS 3 + 3 in 5 cores, repeat biopsy, |
| | | | | 2/4 cores, GS 4 + 3 and 1 core, GS 3 + 4. |
| | 38 | 65 | 36(36) | PSA 6.2 ng/ml; biopsy, 1 core, GS 4 + 3; 4 cores, GS 3 + 4. |
| | 39 | 75 | 40(40) | PSA 5.3 ng/ml; biopsy, 5 cores, GS 4 + 3; 20-65% tumor. |
| | 40 | 55 | 40(40) | PSA 13.7 ng/ml; biopsy, 2 cores, GS 4 + 3, 65% tumor, and |
| | | | | 4 cores, GS 3 + 4, 40-50% tumor. |
| | 42 | 57 | 40(40) | PSA 9.5 ng/ml, biopsy; 10/10 cores, GS 3 + 4, |
| | | | | 35-80% tumor. |
| | 44 | 66 | 32(31) | PSA 4.2 ng/ml; biopsy, 3 cores, GS 3 + 3 in 5, 40, and |
| | | | | 25% tumor, and 2 cores, GS 3 + 4, 40% tumor. |
| | 45 | 64 | 32(31) | PSA 5.4 ng/ml; biopsy, 2 cores, GS 4 + 3; and 5 cores, |
| | | | | GS 3 + 4. |

Numerical Binary Encoding of Bimodally Expressed Genes Can Specify Subpopulations of Exfoliated Prostate Cells.

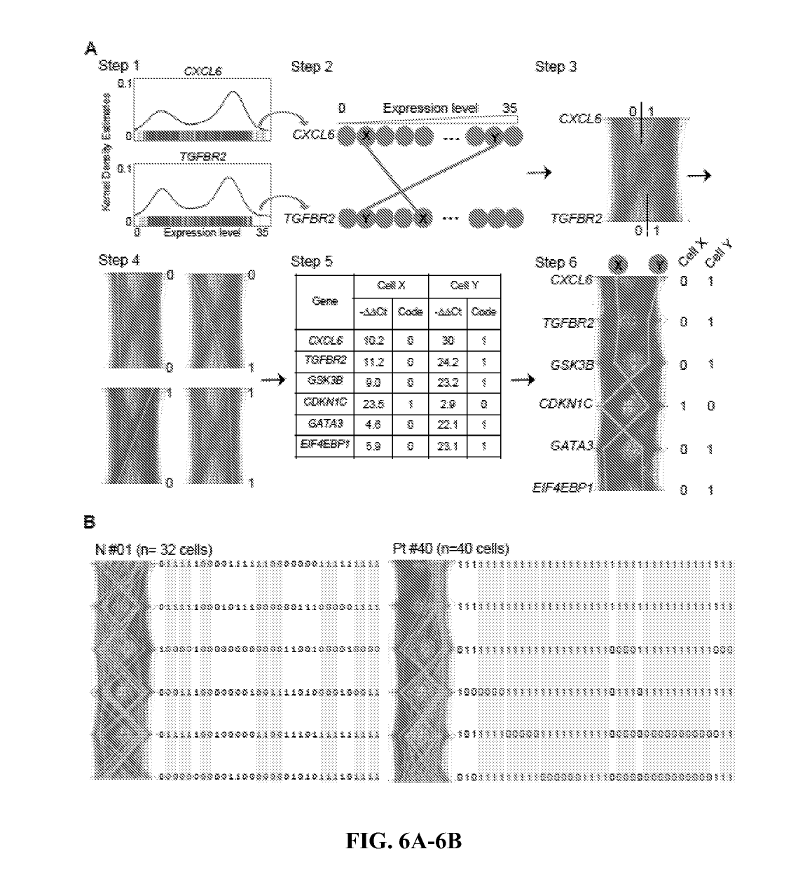

While somatic mutations are frequently used to mark a clonal population of cancer cells (i.e., genotypic clonality), expression patterns of surrogate genes have also been applied to track a clonal process (i.e., phenotypic clonality; Chen and Prchal, (2007) Blood 110, 1411-19). The inventors used these bimodal genes to determine clonal patterns of exfoliated prostate cells in a urine sample. Two or more cells sharing a concordant expression pattern of these genes are considered to belong a clonal population. In order to visualize concordant patterns of gene expression in single exfoliated prostate cells, parallel coordinate plots were constructed for normal controls and patients with BPH, HGPIN, and PCa. Each parallel coordinate plot is comprised of points and connecting lines (see CXCL6 and TGFBR2 as examples in FIG. 6A). The points on an axis, referring to individual cells (total 1220), were aligned from left to right for each gene according to its expression levels from the lowest (0) to highest (35). Expression lines of single cells for a patient were highlighted (FIG. 6B, lower-left). Two parallel lines between two axes suggest a clonal relationship between two cells. When lines intersect in X-shapes, it represents a negative relationship between two cells. This parallel tracking and mapping system was used to identify different clonal populations of exfoliated prostate cells in a patient.

Building on this binary-coding concept, the inventors digitized bimodal expression values with 0 as a low and 1 as a high expression, respectively, in six bimodally expressed genes (see digitizing expression levels into binary codes in Supplemental Experimental Procedure). When arranging the six genes in the order CXCL6-TGFBR2-GSK3B-CDKN1C-GATA3-EIF4EBP1, we identified all 64 (26) combinatorial patterns in 1220 single cells analyzed (e.g., 000100 and 111011 in FIG. 6A, lower-right). For instance, 23 combinatorial patterns were found in 32 single cells of a normal control, #01, indicating that some of these cells shared the same binary codes (FIG. 6B, left). If each binary code (either 1 or 0) were randomly combined in a cell population, the probability would be less than 0.0001 (1/64×1/64) for two given cells to share the same numerical code. Therefore, two or more cells sharing the same combinatorial code in a patient, defined here as clonally-associated numerical pattern (CANP), are very unlikely to happen purely by chance in our analysis, but rather belong to a subpopulation of cells. In this regard, we identified 8 CANPs in this normal individual and 7 CANPs in a patient with PCa (Pt #40; FIG. 6B). When compared to the normal individual (total 23), this patient had a reduced number of combinatorial codes (total 13), suggesting the presence of predominant clonal populations in the urine sample itself. Among the 7 CANPs identified in this patient, 111100 represented the major clonal population (25%) of 40 exfoliated prostate cells analyzed. These cells sharing the same code (111100) are most likely to represent a pathologically hyperproliferative clone in the patient.

Unique Clonally-Associated Numerical Patterns (CANPs) Can Differentiate Aggressiveness of Exfoliated Prostate Cell Subpopulations.

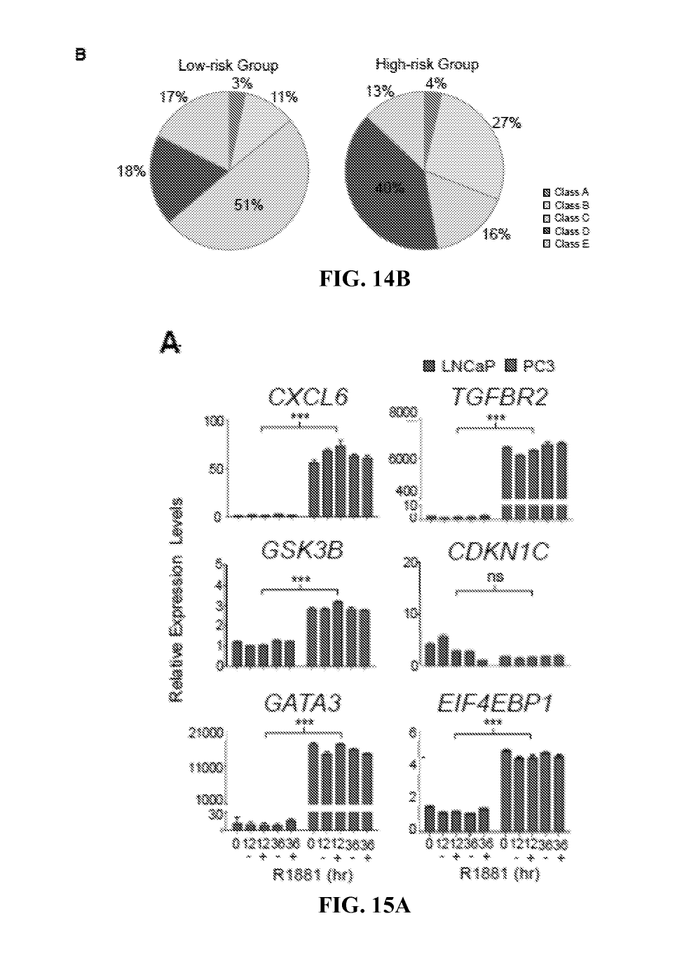

Single-cell expression patterns of genes that can be connected in a string for a given cell. A patina line traces an expression path of a cell across six genes. Connectivity paths are converted to binary code-strings with 0 for low and 1 for high expression, respectively. Clonally-associated numerical patterns (CANPs) can correspond to classes in FIG. 14 (A): Class A (n=7), Class B (n=7), Class C (n=16), Class D (n=8), and Class E (n=12). To use the CANP system for clonal analysis, a total of 38 parallel coordinate plots were generated. There were 832 clonal cells, sharing CANPs in individual patients, in a total of 1220 exfoliated prostate cells isolated from normal controls, BPH, and two subgroups of PCa patients without undergoing hormone ablation or related therapies. PCa subgroups are (1) low-risk (including HGPIN) patients with GS<3+4 with small biopsy tumor volume (5-30%) and (2) highrisk patients with GS≧3+4 with large biopsy tumor volume (>30%; Table 2). Two additional patients, #17 and #19, who had distant metastasis and/or extremely high serum PSA values, might have received therapies prior to their urine collections. Their exfoliated cells were excluded from the analysis because original clonal profiles of these patients might have changed as a result of receiving therapies.

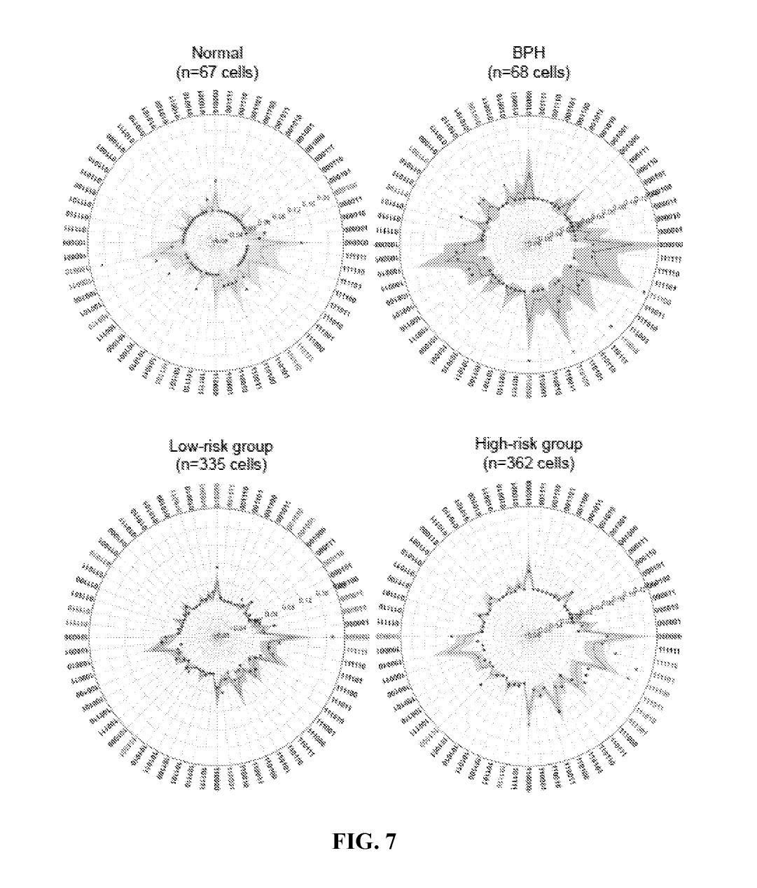

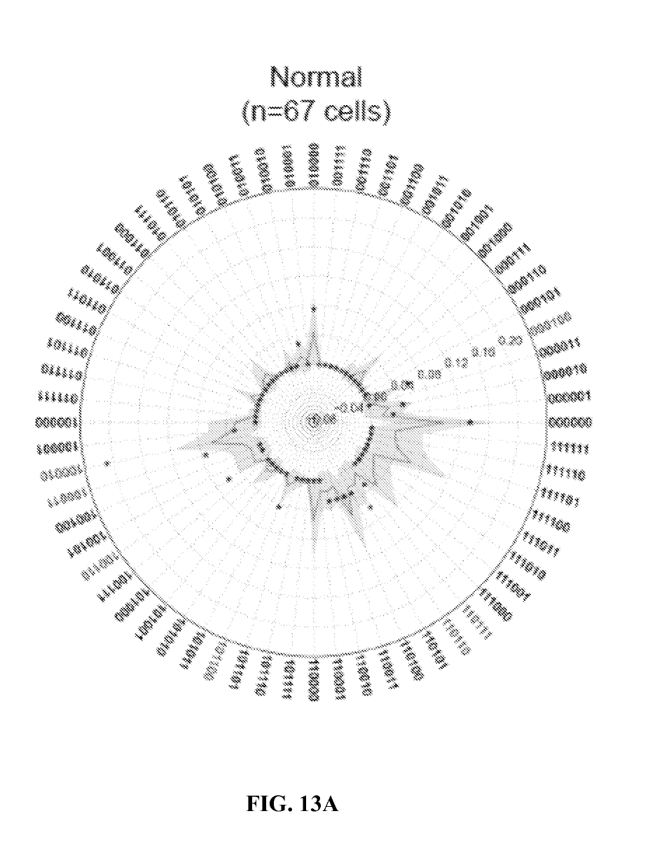

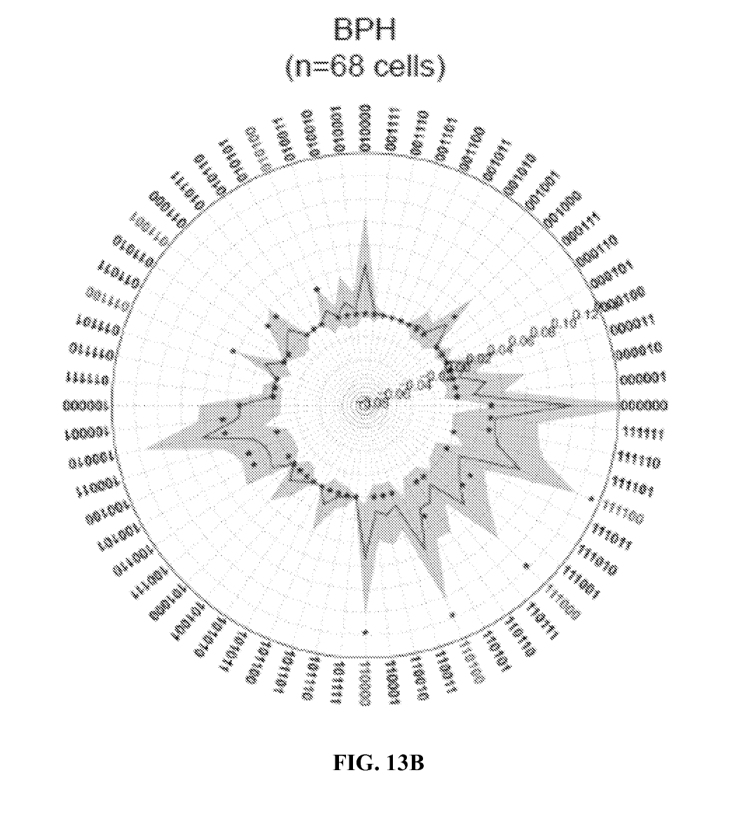

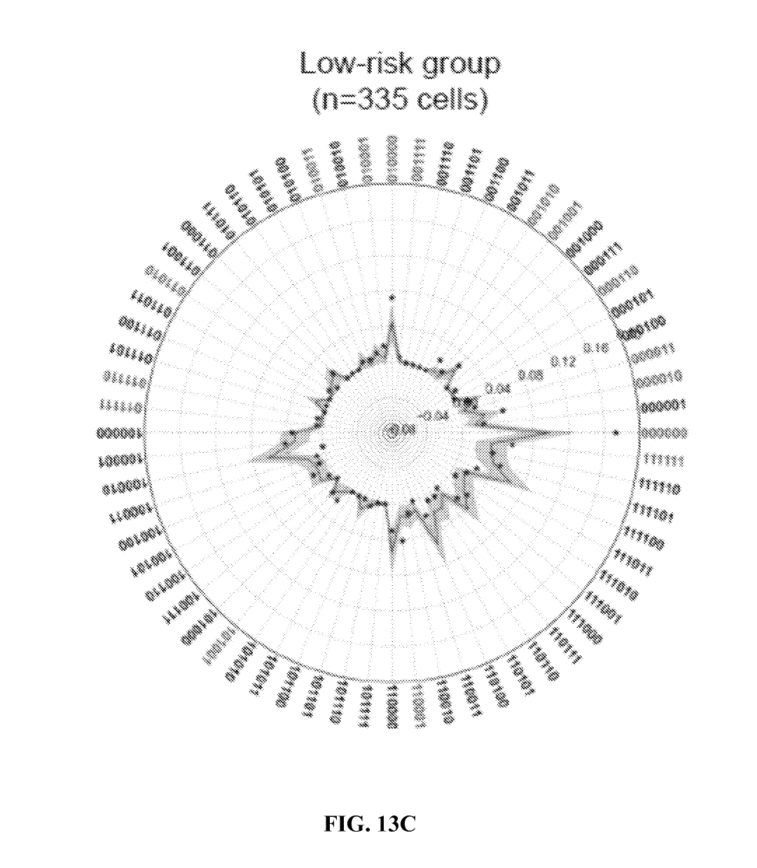

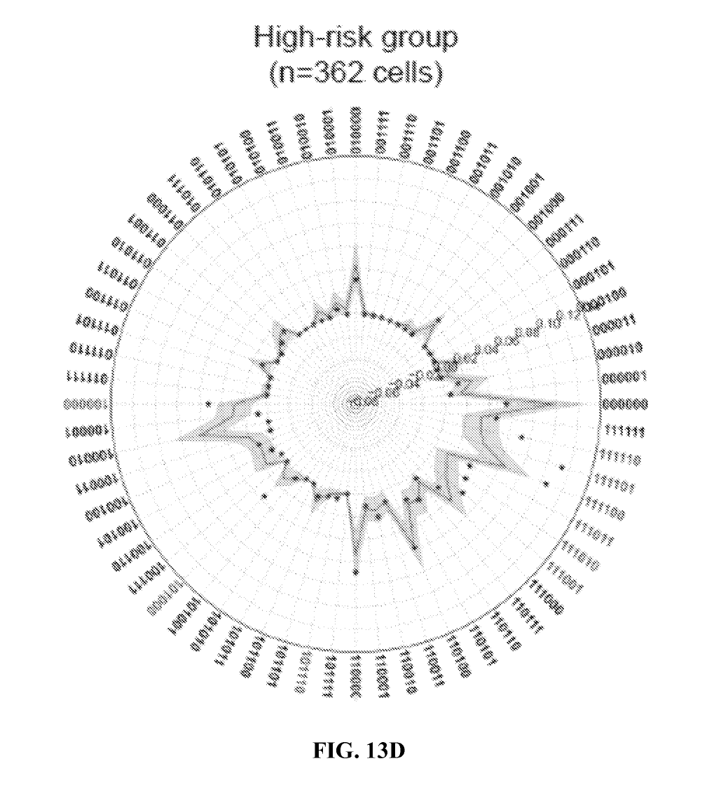

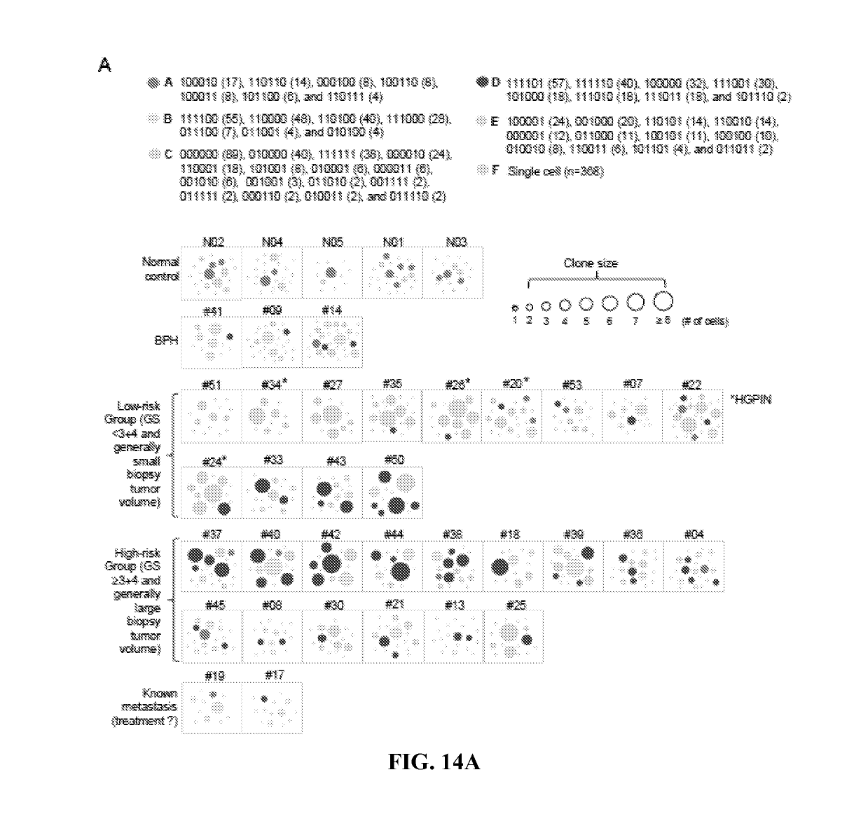

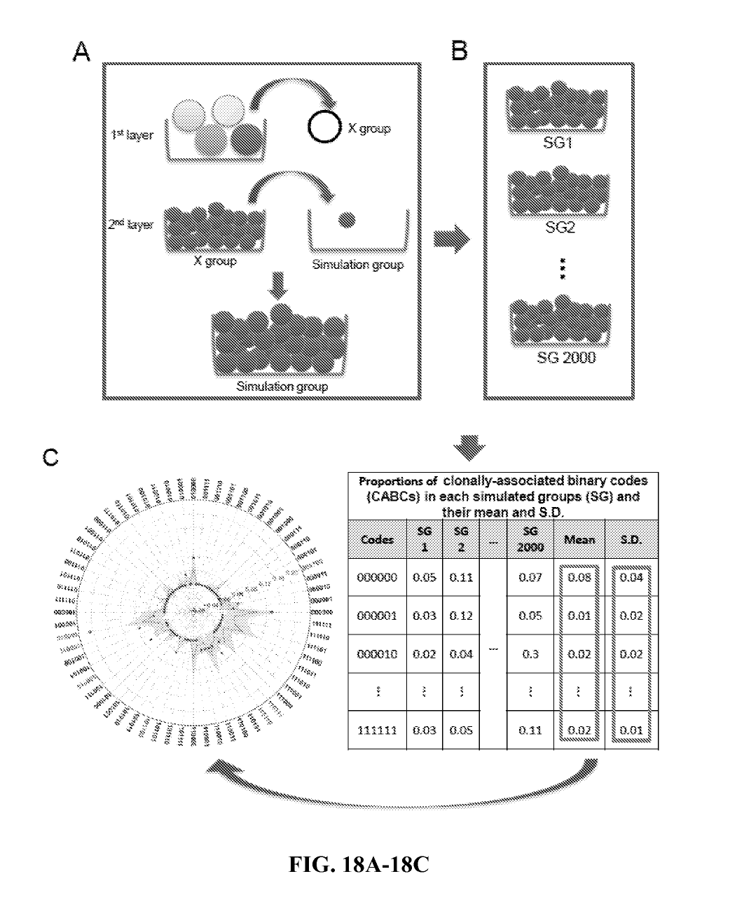

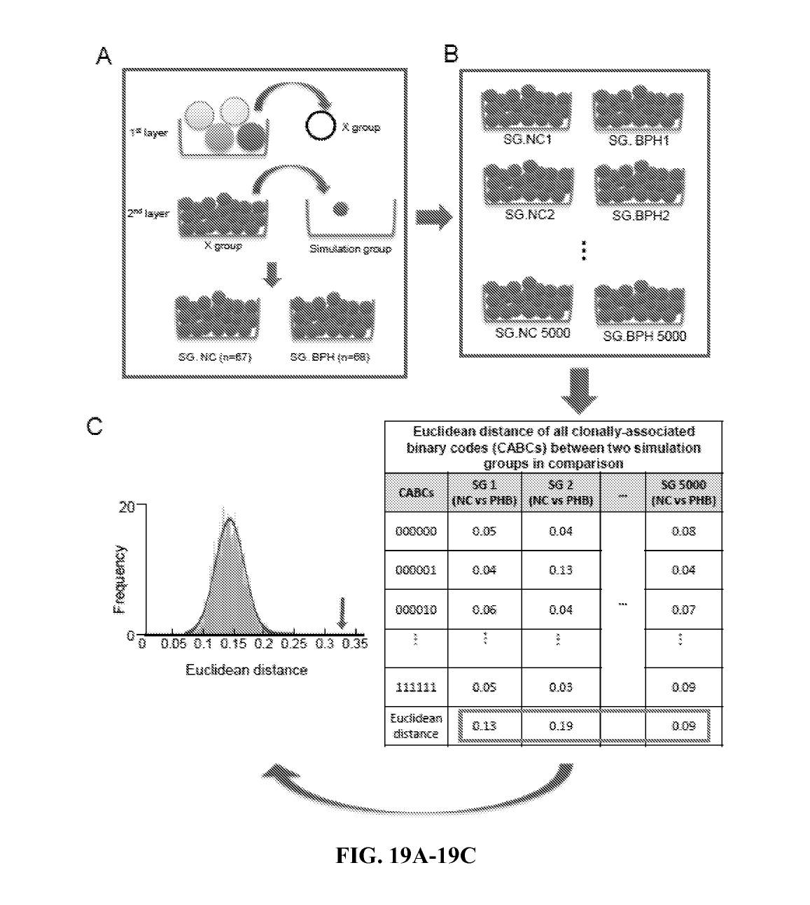

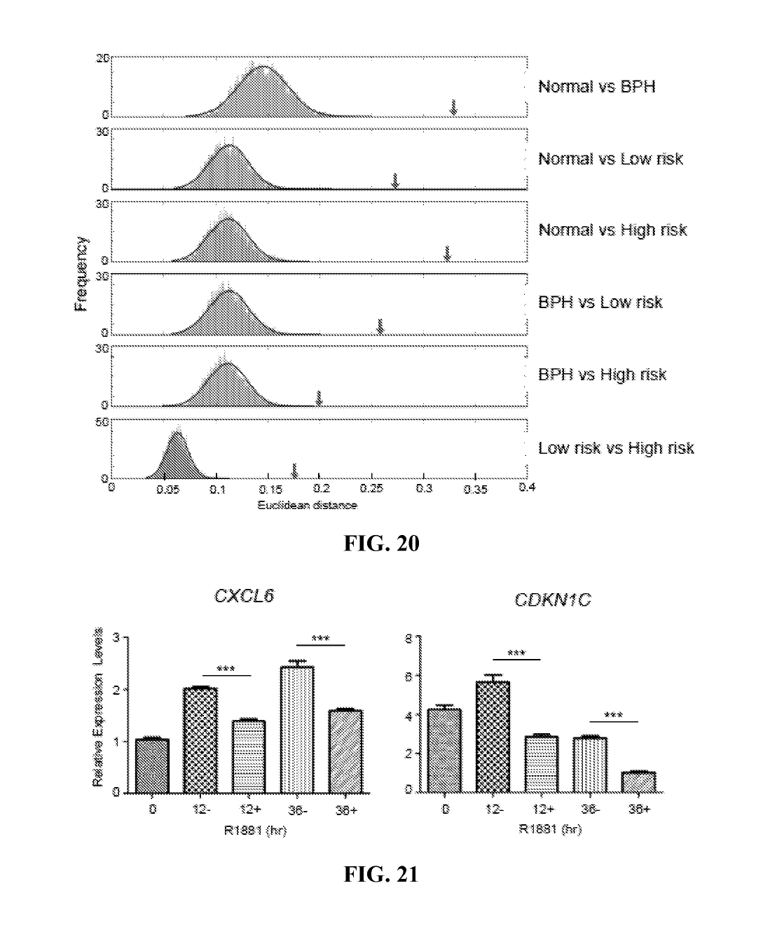

A permutation resampling test was performed 2000 times to identify unique CANPs in 832 clonal cells associated with subpopulations of exfoliated prostate cells in normal controls and three patient groups (FIG. 18). The results were shown in polar plots, displaying the average value of the proportion of all combinatorial codes as the solid line and 1.7× standard deviation 9 as the shadow span from 2000 simulation groups (FIG. 7 and FIG. 13). This computational modeling identified 38 unique CANPs whose presence in the four existing groups were significantly higher than those in the simulation groups (p<0.05 in one-tailed test). The finding of the 38 CANPs was independently validated in the Euclidean distance analysis, confirming that these clonal codes can distinguish cell populations between any two patient groups (p<0.0001; FIG. 19 and FIG. 20).

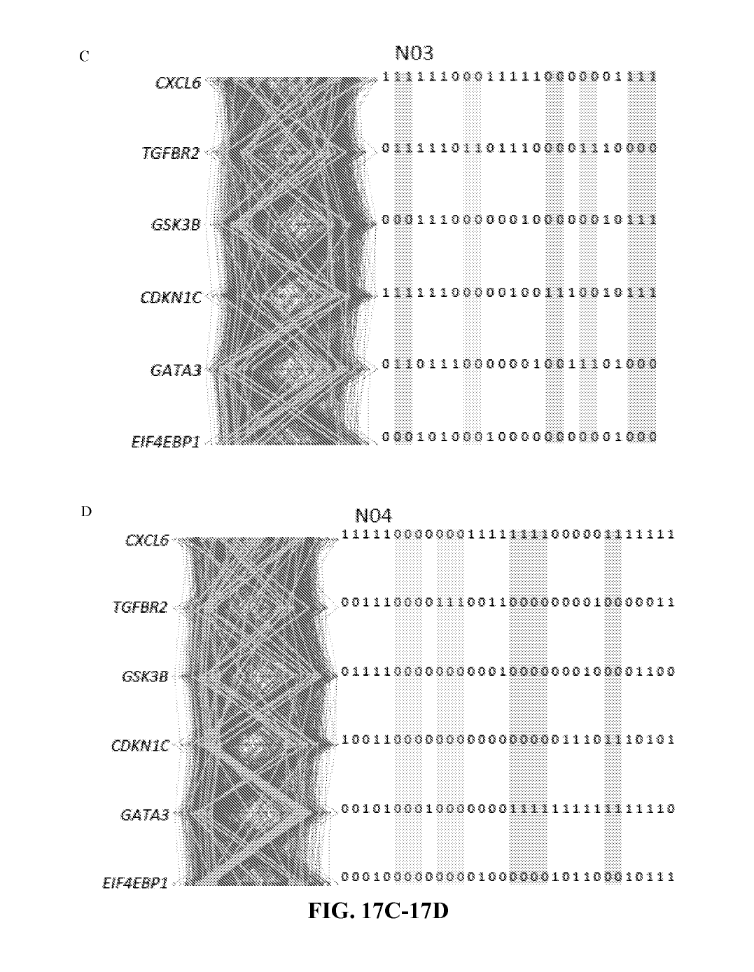

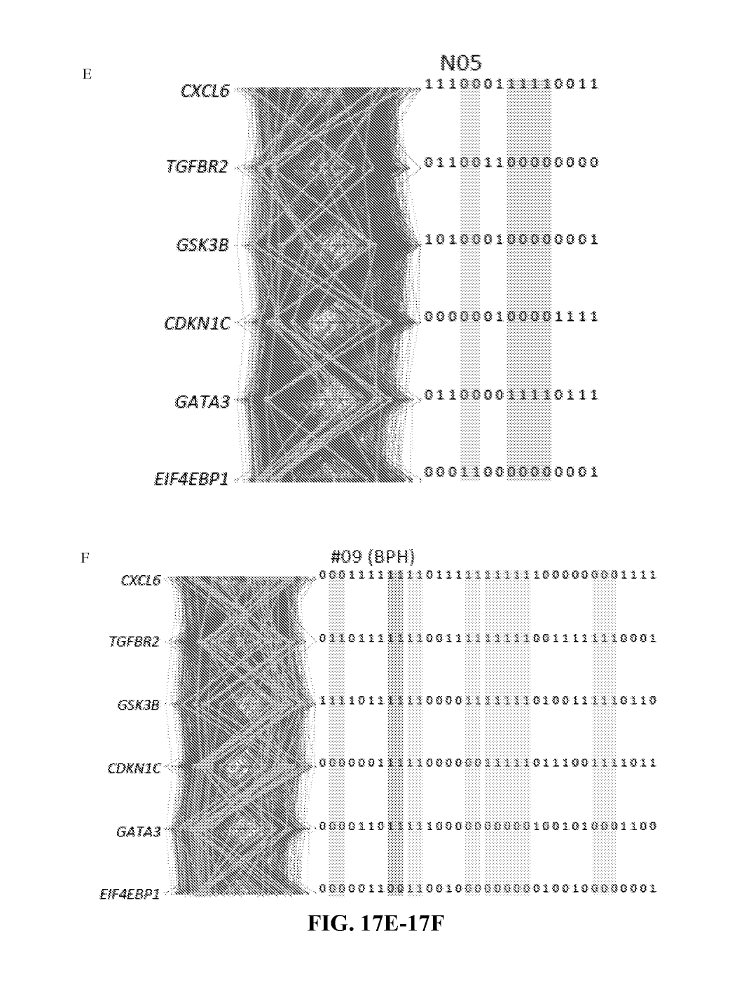

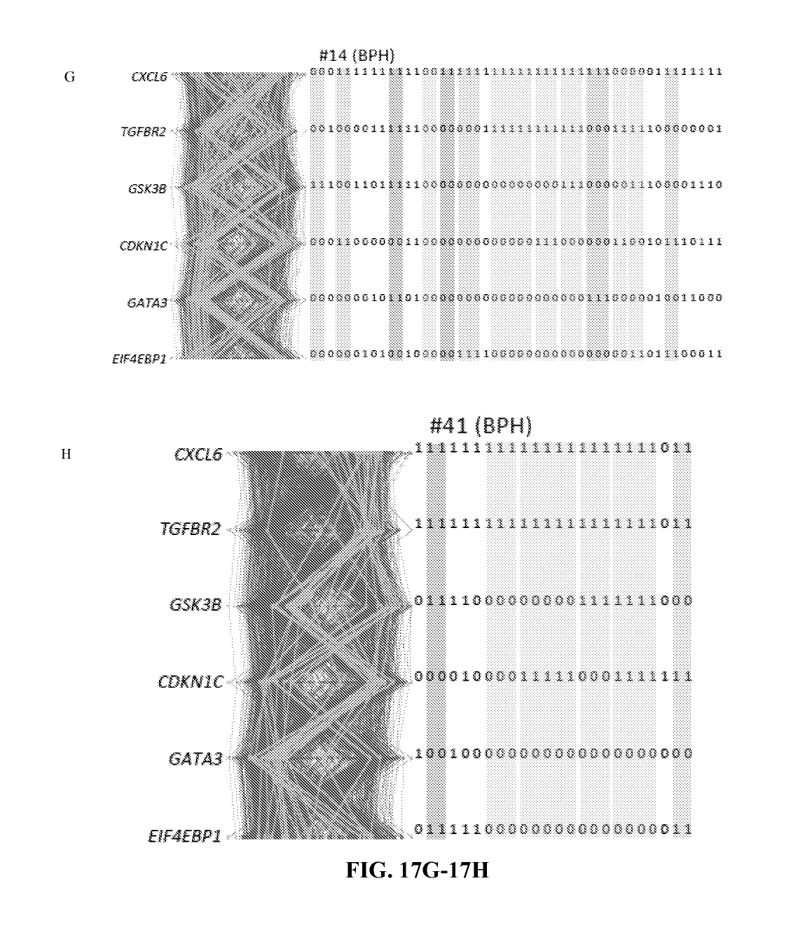

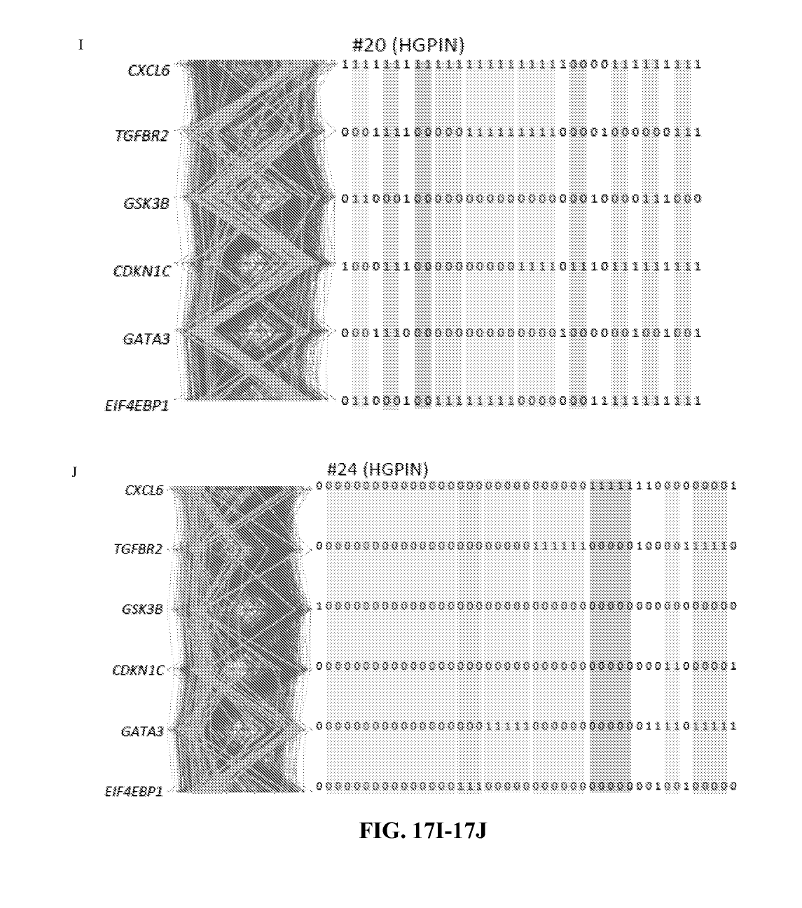

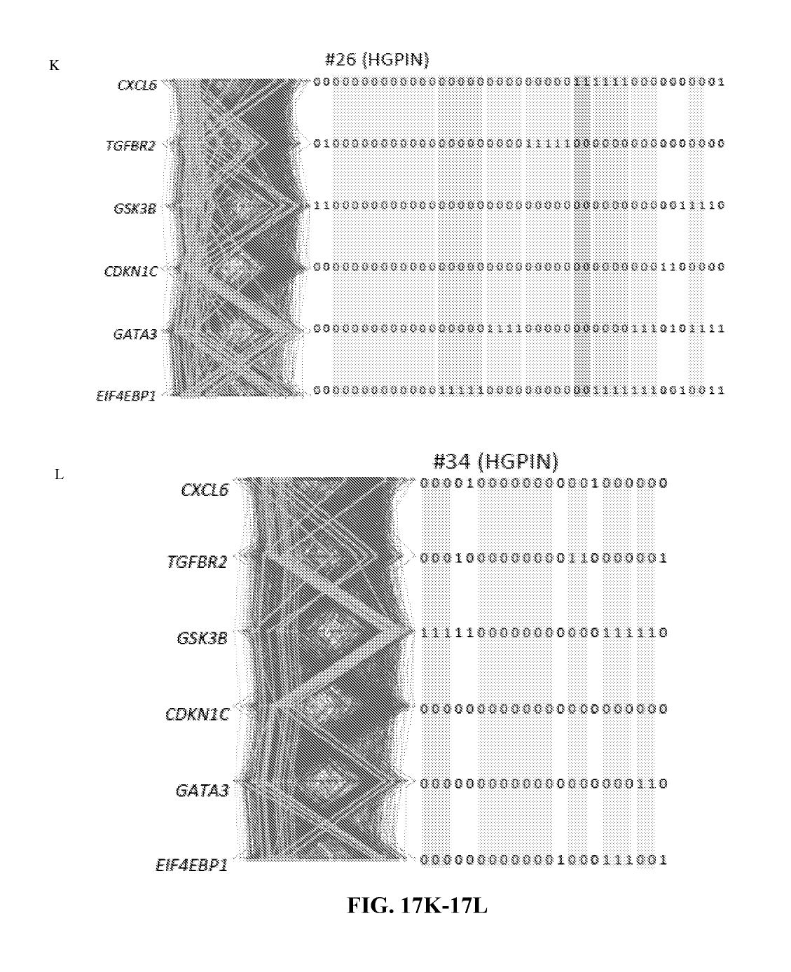

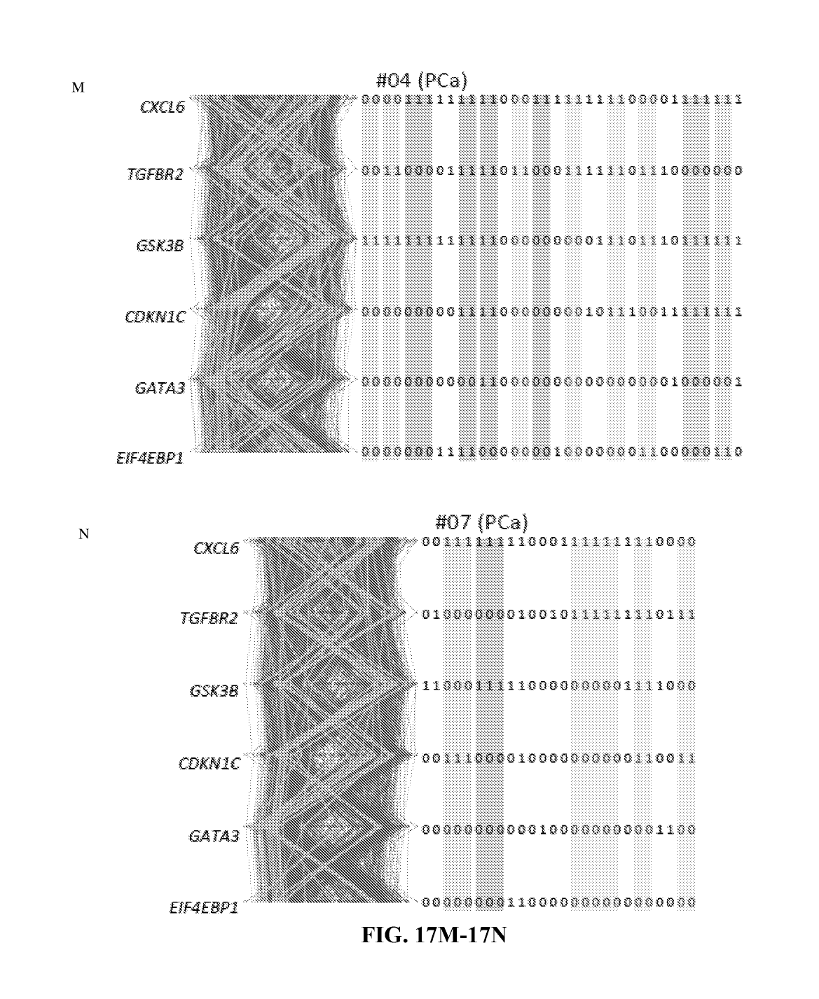

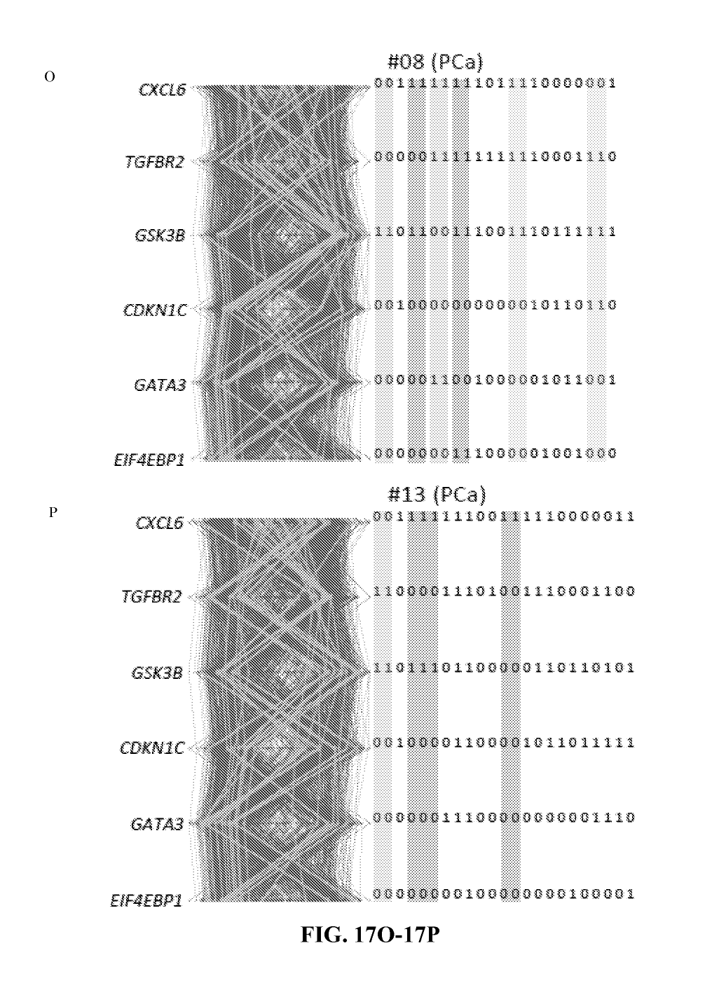

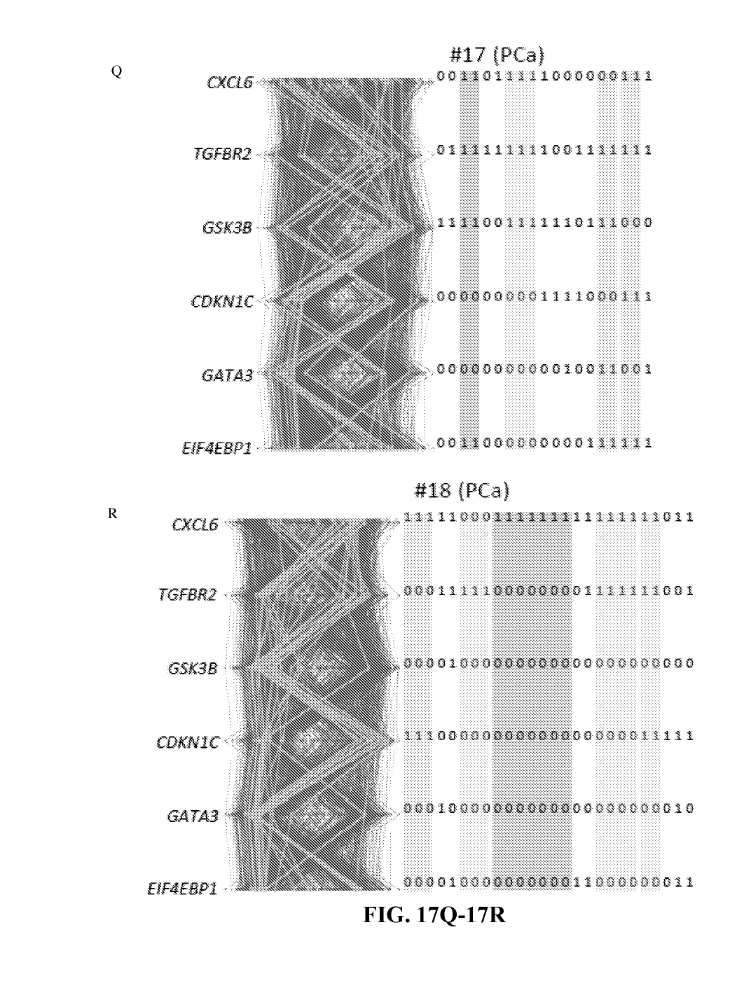

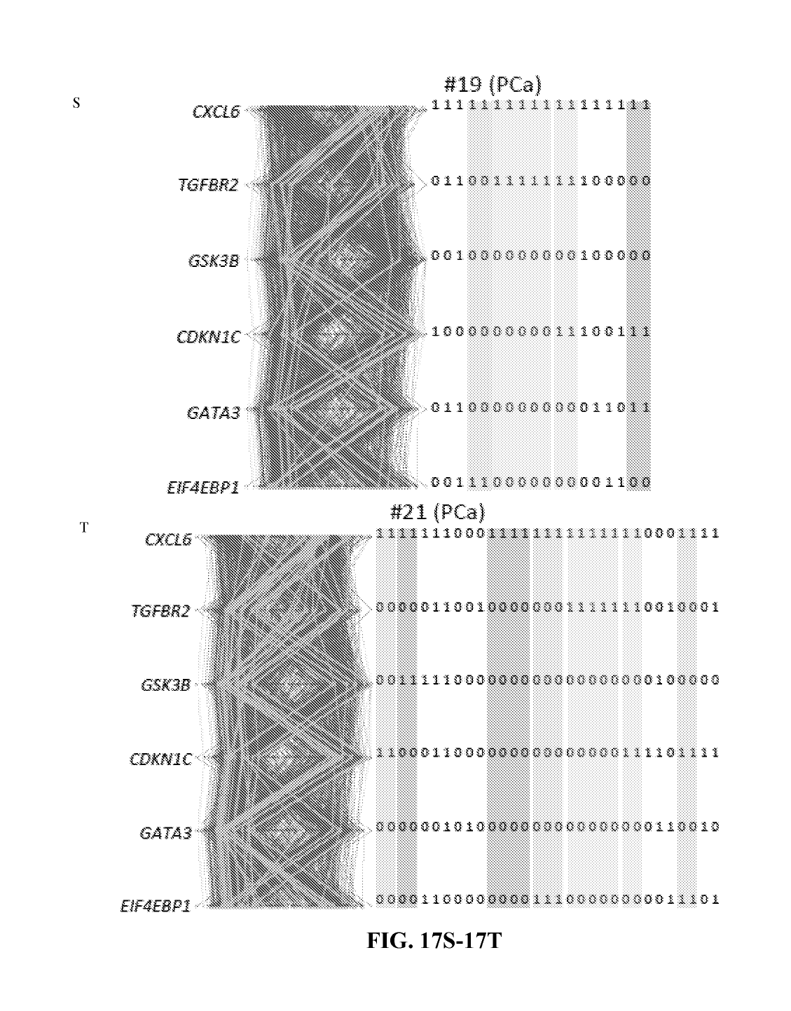

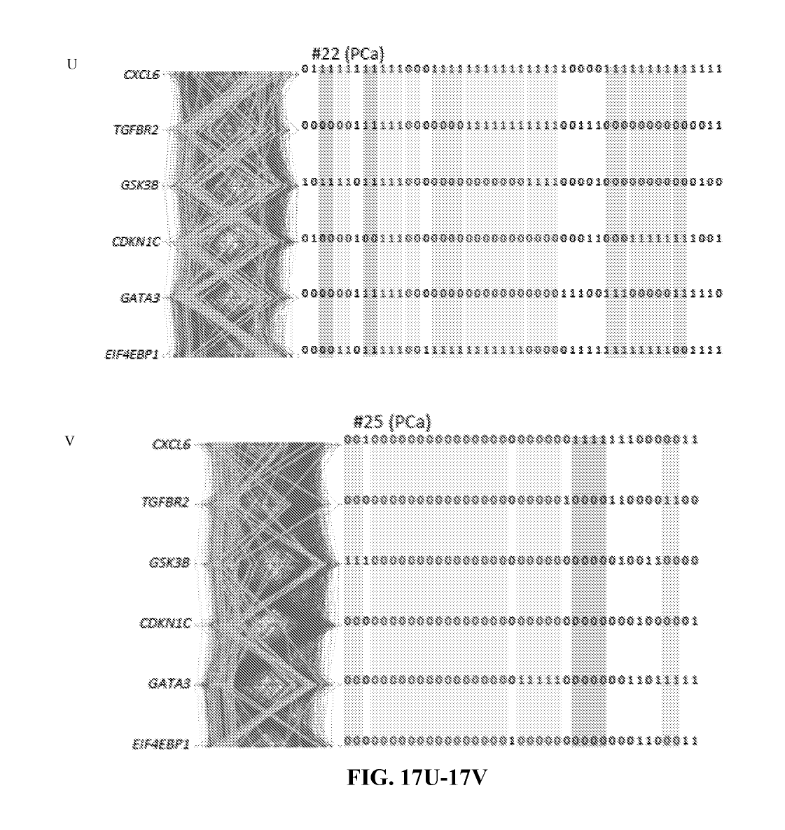

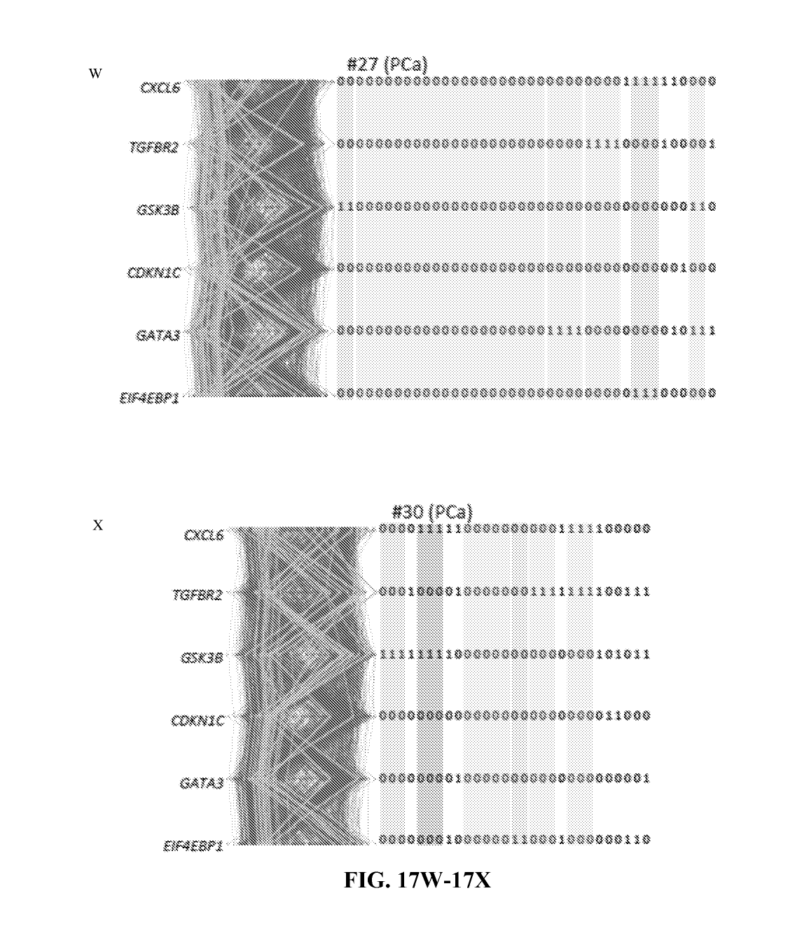

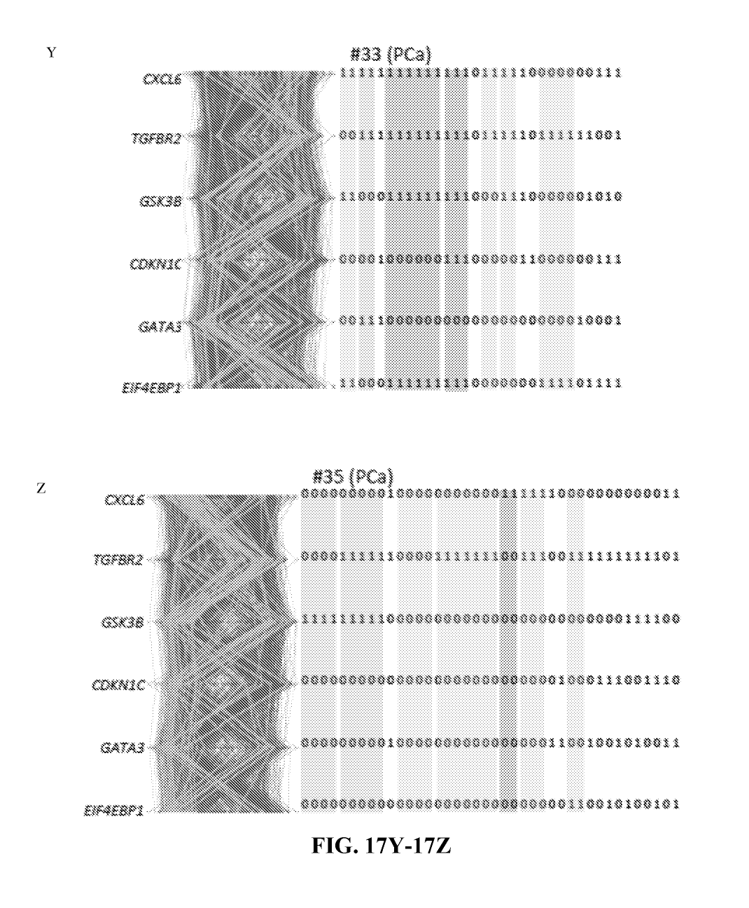

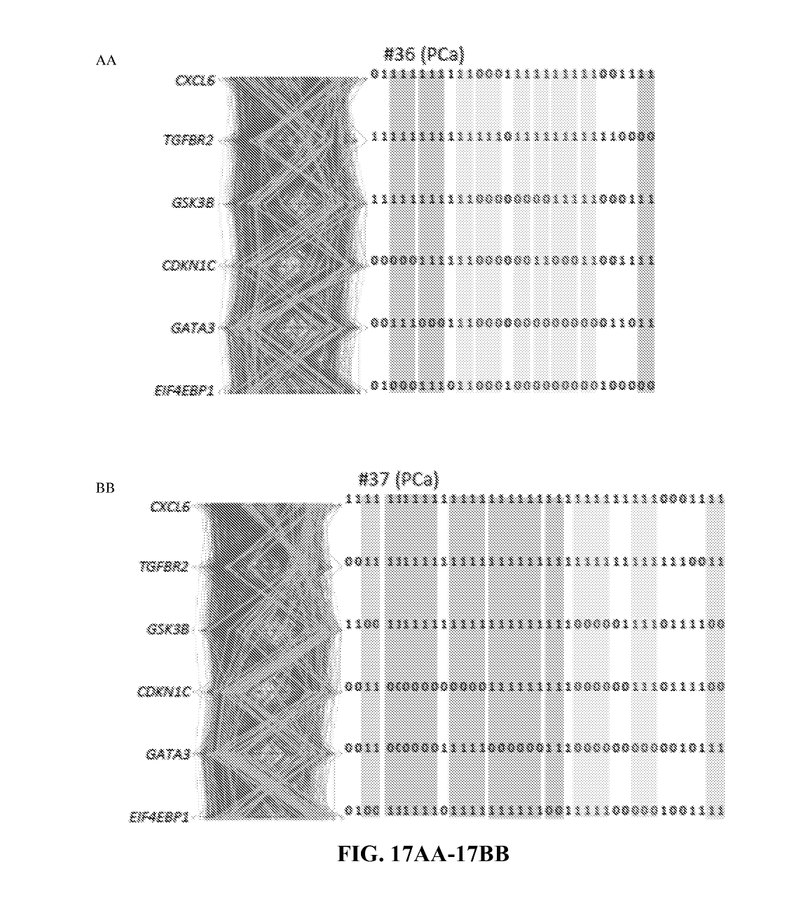

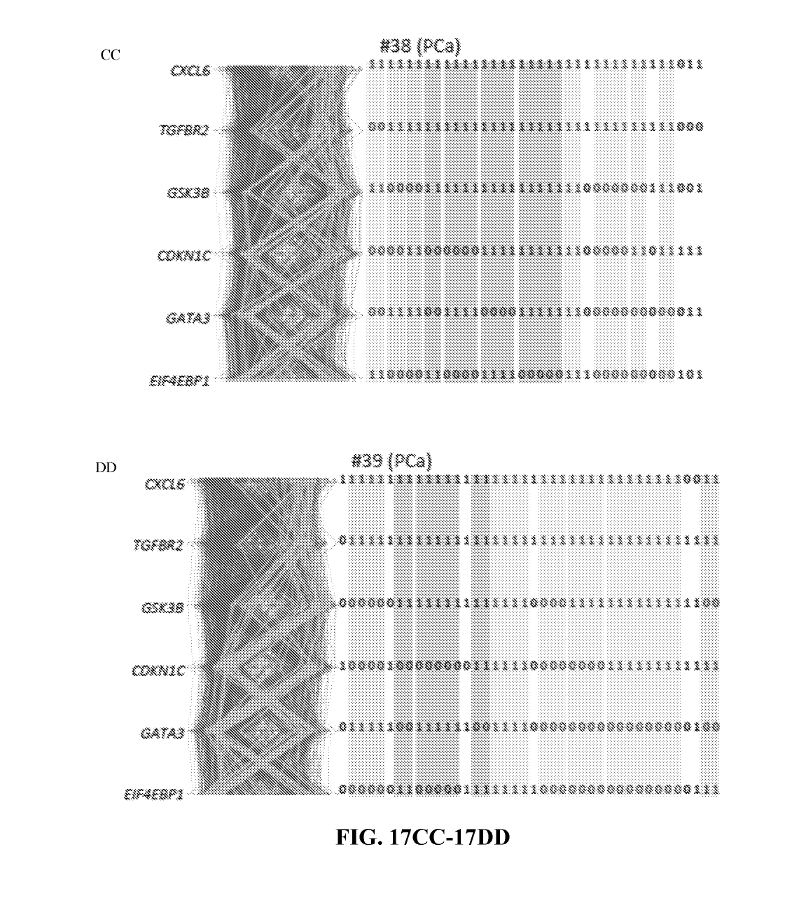

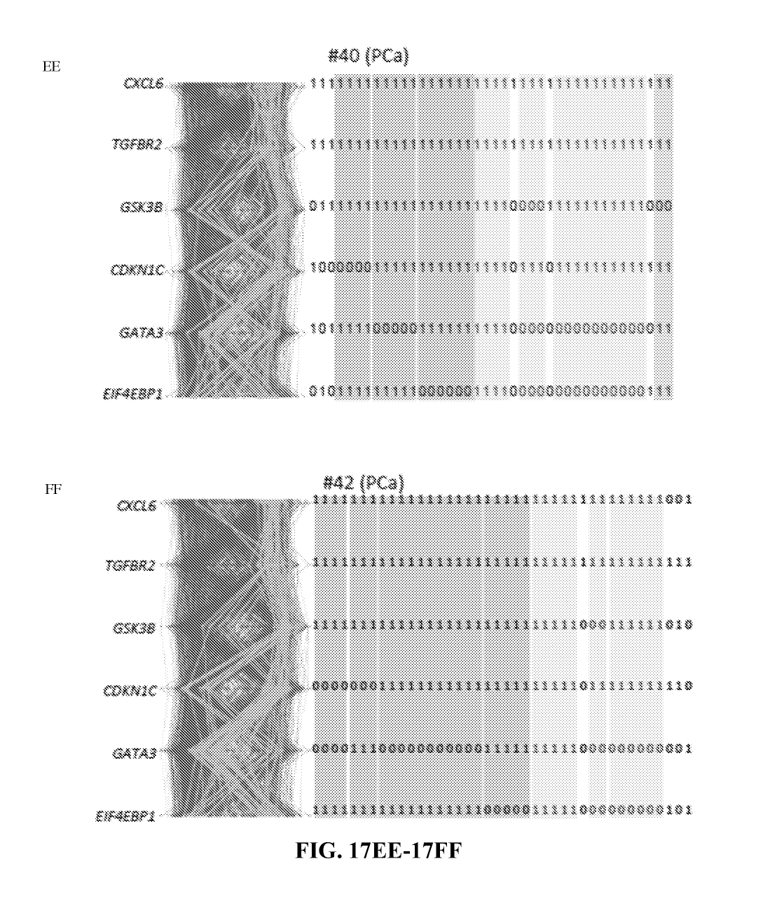

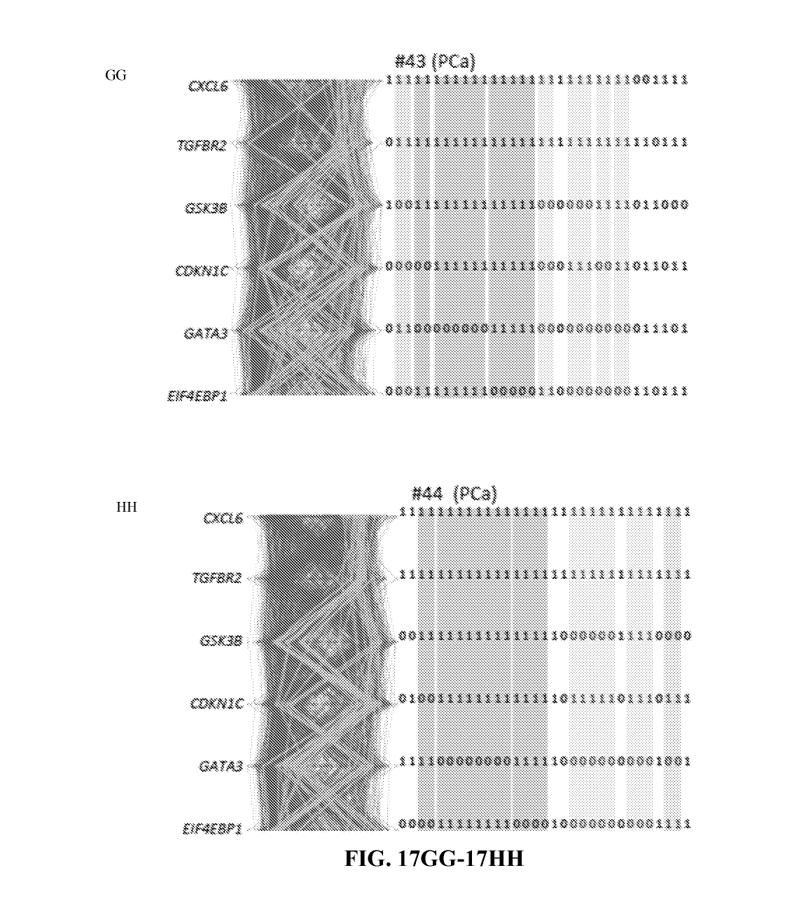

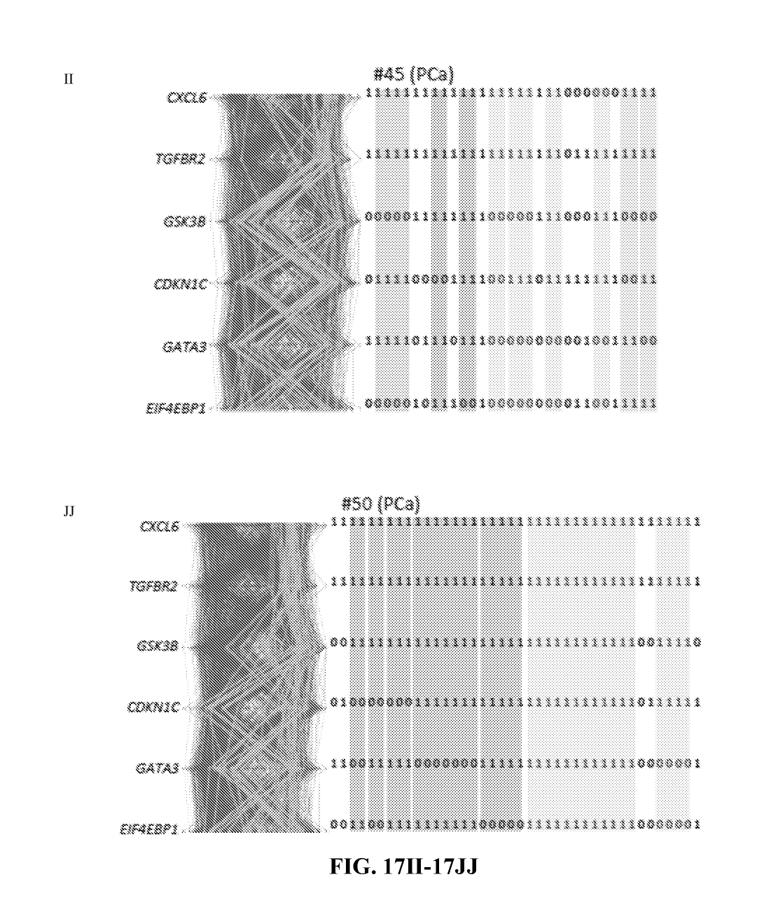

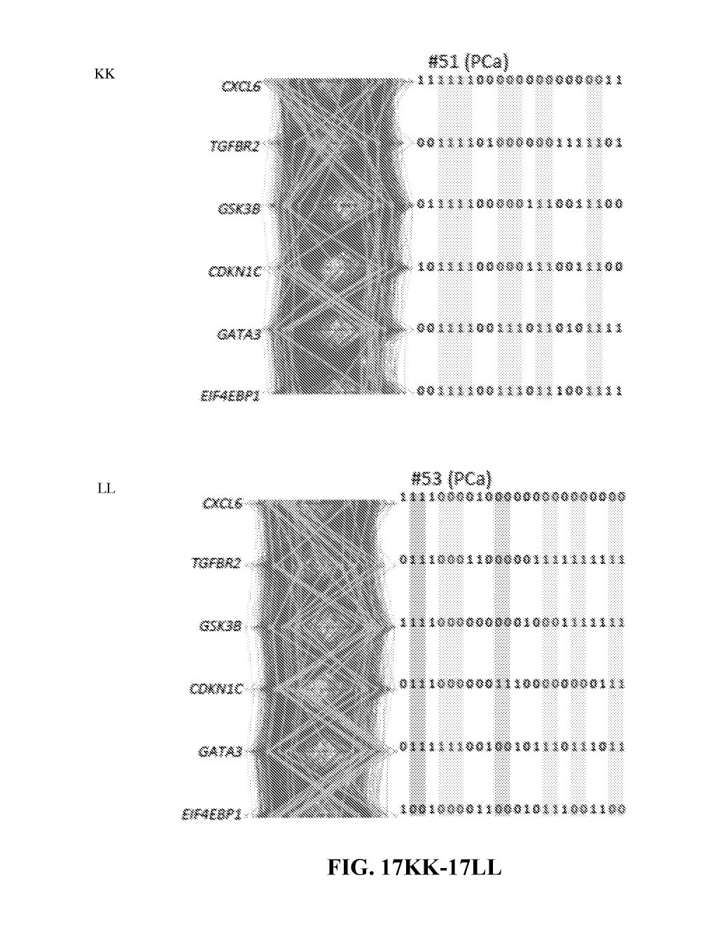

Different classes of CANPs and their corresponding clonal sizes were assigned to normal controls and patients (FIG. 14A and FIG. 17). Class A CANPs (n=7) were mainly present in normal controls, although these combinatorial codes were sometimes observed in urine samples of BPH and PCa patients (Pt #04, 14, 20, 22, 37, 38, 40, and 45). Class B CANPs (n=7) were not only found in all BPH patients (n=3) but also present in at least 71% (n=20) of 28 PCa patients. These clonal codes are likely associated with non-cancerous hypertrophy of the prostate gland, which is commonly seen in PCa patients. In addition, two of these CANPs—111100 or 110000 were frequently present in large clonal populations (n≧5 cells) of 5 PCa patients (i.e., #7, 21, 39, 40, and 42). Whether these large clonal populations in urine are associated with extremely enlarged prostates remains to be determined in PCa patients.

Class C CANPs (n=16, pink) represented dominant clonal populations in low-risk PCa patients (51% of 335 clonal cells), but were less frequent in high-risk PCa patients (16% of 362 clonal cells; FIG. 14B). Since four of the 16 codes (i.e., 000000, 000010, 010000, and 110001) were frequently present in large clonal subpopulations (n≧5 cells) of HGPIN patients, these CANPs were likely originated from precursor lesions that may progress into malignant clones in PCa patients (Marusyk and Polyak, 2013). Class D CANPs (n=8) were preferentially present in high-risk patients (40% of 362 clonal cells), but appeared less frequent in the low-risk group (19% of 328 clonal cells; FIG. 14B). Taken together, these 24 clonal codes (Class C and D) could be putative biomarkers for differentiating high-risk from low-risk PCa.

Interestingly, four patients (#24, 33, 43, and 50) with low-risk PCa on biopsy appeared to have 1-2 large clonal populations (≧4 cells per clone) of exfoliated cells carrying Class D CANPs. As higher-grade PCa is often subsequently confirmed when patients with lower-grade tumors undergo repeat biopsy or radical prostatectomy (Chun et al., 2006; Park et al., 2013), further follow-up of these individuals may confirm the presence of more aggressive tumors. Class E CANPs (n=12) represented clonal populations that were commonly found in all patients and could not be used to stratify different risk groups. As mentioned earlier, patients #17 and 19 who had aggressive PCa with distant metastasis (e.g., bone) and/or extremely high serum PSA values had only smaller clonal populations of exfoliated prostate cells in their urine samples. The result suggests the potential of this non-invasive, single-cell approach to monitor patients' response to treatment.

The CANP 111011 is Functionally Linked to an Adverse Phenotype of Prostate Cancer Cells.

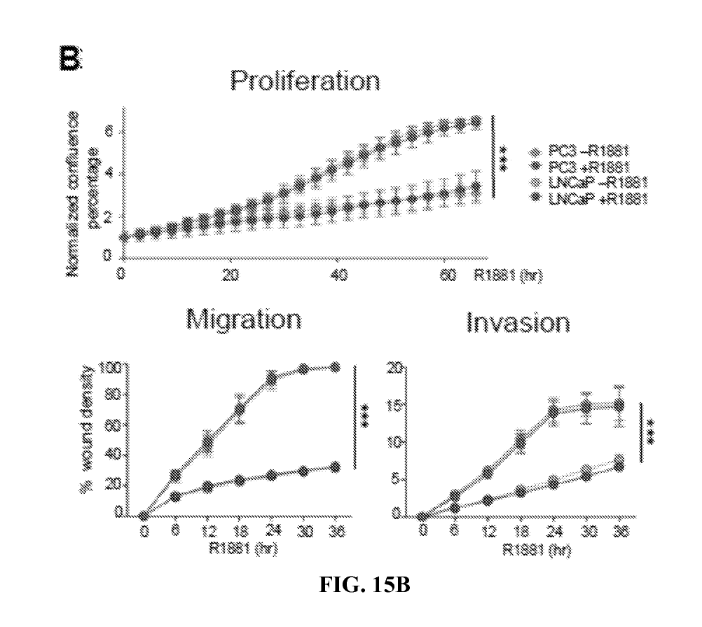

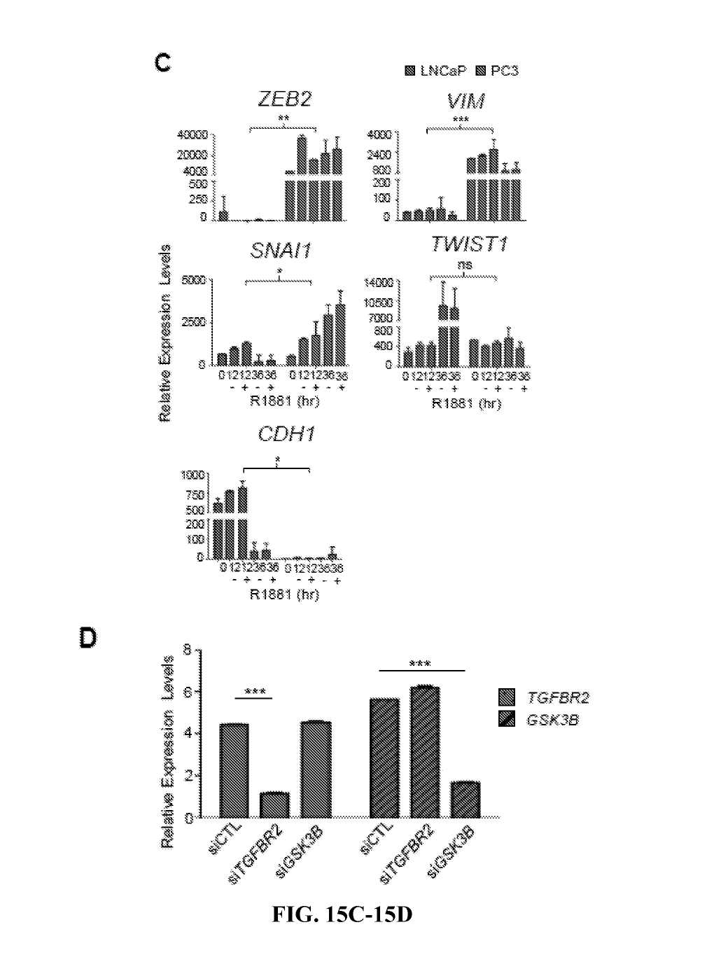

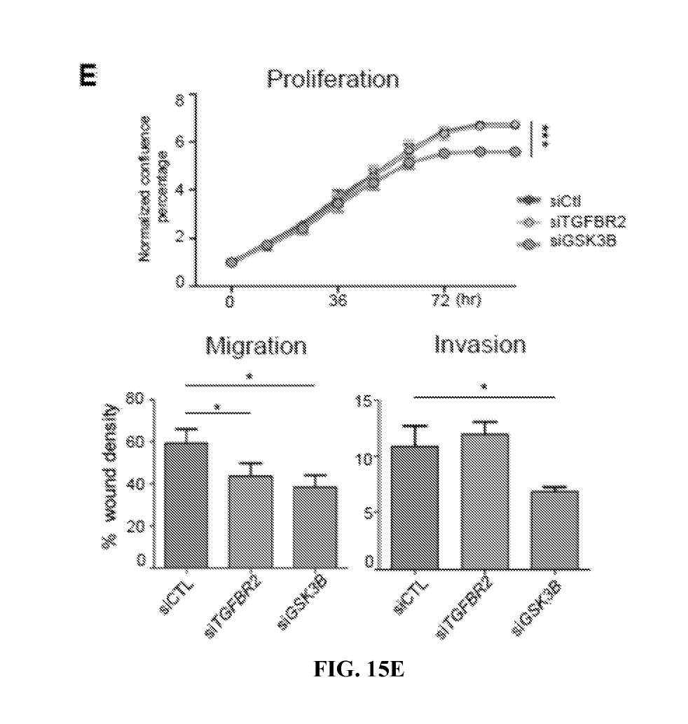

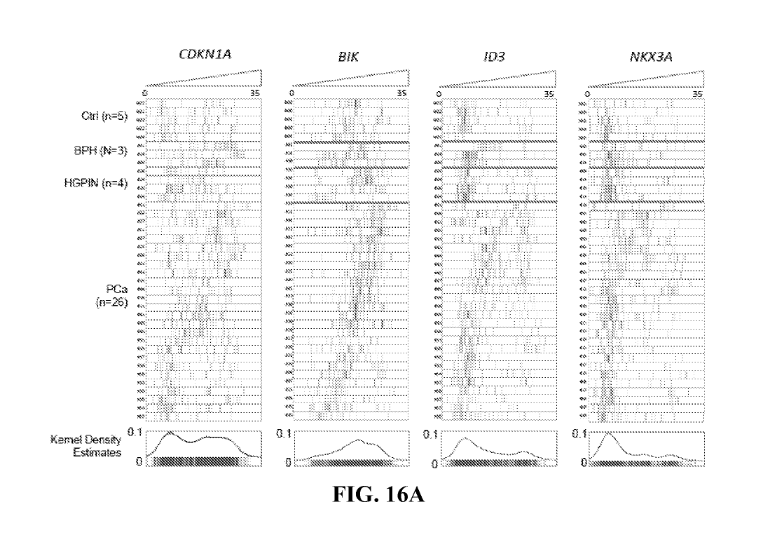

To establish a causal link between CANPs and PCa progression in vitro, expression analysis of the aforementioned six genes were conducted in prostate cancer cell lines (FIG. 15A). Expression patterns of the six genes in two cell lines, LNCaP and PC3, were found to highly resemble 000000 (low-risk code) and 1011011 (high-risk code), respectively. Five of the genes—CXCL6, TGFBR2, GSK3B, GATA3, and EIFEBP1 were concordantly down-regulated (i.e., coded as 0) in LNCaP cells while highly expressed (i.e., coded as 1) in PC3 cells. The expression of the other gene, CDKN1C, was known to be repressed (coded as 0) in both cell lines (FIG. 15A, middle-right). All the experiments were also carried out in the presence or absence of androgen analogue, R1881 (1 nM), to evaluate the effects of androgen signaling on altered expression of the six genes. The expression of CXCL6 and CDKN1C was further repressed in R1881-treated LNCaP cells relative to untreated cells for a period of 36 hrs (FIG. 21). This androgen stimulation at a low-dose condition, however, did not cause noticeable changes in cell growth, migration and invasion of LNCaP cells (FIG. 15B). As expected, this treatment did not alter gene expression patterns or cellular phenotypes in androgen-independent PC3 cells (Singh et al., 2008). When compared to LNCaP cells, PC3 cells appeared to have a more aggressive phenotype with faster cellular proliferation, migration and invasion (p<0.001; FIG. 15B). In addition, this phenotype (i.e., 111011) was associated with a more advanced profile of epithelial-mesenchymal transition (i.e., three up-regulated markers—ZEB2, VIM, and SNAI1 and one down-regulated marker—CDH1) in PC3 relative to LNCaP cells (FIG. 15C).

To investigate if altered expression of these bimodal genes is contributing directly to the aggressiveness of PC3 cells, we performed siRNA knockdown of two representative genes, TGFBR2 and GSK3B. These two loci were chosen because of their involvement in modulating TGF-b signaling for PCa progression. Transient knockdown of TGFBR2 and GSK3B moderately attenuated aggressive behaviors of PC3 cells in migration (FIGS. 15D and 15E). However, only decreased GSK3B expression suppressed proliferation and invasion of PC3 cells. This initial phenotypic assay suggests that the high expression of the two genes offers more than being predictive bystanders and is functionally involved in advanced development of PC3 cells and likely in high-risk PCa.

1. A method for stratifying subpopulations of single cells comprising:
(a) measuring single cell gene expression levels for a plurality of target cells isolated from a sample;
(b) transforming the single cell gene expression levels to binary form based on bimodal distribution analysis forming a clonally associated numerical pattern (CANP) for each individual target cell; and
(c) classifying the sample based on comparing the distribution of CANPs with reference CANPs.

2. The method of claim 1, wherein the gene expression levels is determined using cycle threshold amplification.

3. The method of claim 1, further comprising representing the CANPs as a graphical display.

4. The method of claim 3, wherein the graphical display is a polar plot where the lines of the plot represent a proportion of a CANP to all CANPs with a shadow covering a predetermined standard deviation.

5. The method of claim 1, further comprising validating the CANPs using a Euclidean distance analysis.

6. The method of claim 1, wherein the sample is a biological fluid or dispersed tissue sample.

7. The method of claim 6, wherein the sample is a urine sample.

8. The method of claim 7, wherein the urine sample is obtained within a week of a digital rectal examination.
